(19) United States
(12) Patent Application Publication
(10) Pub. No.: US 2026/0103453 A1
(43) Pub. Date: Apr. 16, 2026

(54) NOVEL PYRIMIDINE DERIVATIVE AS BRUTON'S TYROSINE KINASE INHIBITOR

(71) Applicant: HANMI PHARM. CO., LTD., Hwaseong-si, Gyeonggi-do (KR)

(72) Inventors: Sun Young JANG, Hwaseong-si (KR); Seok Jong KANG, Hwaseong-si (KR); Yong Taek LEE, Hwaseong-si (KR); Gun Woo LEE, Hwaseong-si (KR); Kwee Hyun SUH, Hwaseong-si (KR)

(21) Appl. No.: 19/120,716

(22) PCT Filed: Oct. 13, 2023
(86) PCT No.: PCT/KR2023/015876

§ 371 Date: Apr. 11, 2025

(30) Foreign Application Priority Data
Oct. 14, 2022 (KR) 10-2022-0132706

(51) Int. Cl. C07D 403/10; A61K 31/506; A61K 31/517; C07D 239/42; C07D 401/10; C07D 401/14; C07D 403/06

(52) U.S. Cl. CPC C07D 403/10; A61K 31/506; A61K 31/517; C07D 239/42; C07D 401/10; C07D 401/14; C07D 403/06

(57) ABSTRACT

The present disclosure relates to a novel pyrimidine derivative, a solvate or stereoisomer thereof, or a pharmaceutically acceptable salt thereof, and a pharmaceutical composition including any of the foregoing as an active ingredient, the pyrimidine derivative being represented by Formula I and having inhibitory activity against Bruton's tyrosine kinase. The novel pyrimidine derivative of the present disclosure effectively inhibits the enzymatic activity of Bruton's tyrosine kinase (BTK), and thus may be useful as an active ingredient in a pharmaceutical composition for preventing or treating BTK-mediated disease such as autoimmune disease or cancer: [Formula I]

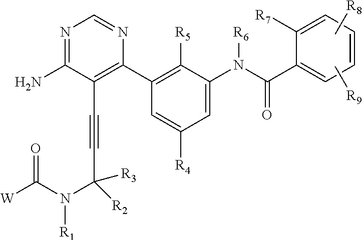

### TECHNICAL FIELD

The present disclosure relates to a novel pyrimidine derivative having Bruton's tyrosine kinase inhibitory activity and a pharmaceutical composition containing the same as an active ingredient.

### BACKGROUND ART

Bruton's tyrosine kinase (BTK), a member of the Tec family of tyrosine kinases, has an essential role in the signaling cascade of receptors, such as B-cell receptors (BCRs), Fc receptors, chemokine receptors, toll-like receptors (TLRs), and CD40, and functions as a regulator of early B-cell development as well as mature B-cell activation, signaling, and survival.

These B cells are signaled by BCRs, which recognize antigens attached to the surface of antigen-presenting cells, and then activated into mature antibody-producing cells. However, abnormal signaling by BCRs cause abnormal proliferation of B cells and formation of pathological autoantibodies, which can lead to cancers, autoimmune diseases, and/or inflammatory diseases.

Therefore, when BTK is inhibited in the abnormal proliferation of B cells, the signaling by BCRs is blocked and B cell-mediated diseases can be blocked accordingly. In this regard, use of BTK inhibitors can be a useful approach for treating various cancers or tumors and B cell-mediated diseases such as autoimmune diseases.

Under such technical background, multifaceted research is underway to develop BTK inhibitors that can effectively inhibit the activity of BTK (WO2008/039218). However, BTK inhibitors that have been studied to date do not exhibit sufficiently selective inhibitory activity against BTK, are resistant to drugs, or have side effects such as toxicity to normal cells, and thus cannot be effectively used for the treatment of various cancers, tumors, and autoimmune diseases.

Accordingly, as a result of research on new compounds, the inventors of the present disclosure identified a novel pyrimidine derivative compound with excellent selective inhibition ability as a BTK inhibitor, thereby completing the present disclosure.

### DISCLOSURE

### Technical Problem

An aspect is to provide a novel pyrimidine derivative that exhibits inhibitory activity against Bruton's tyrosine kinase (BTK).

Another aspect is to provide a pharmaceutical composition for preventing or treating BTK-mediated disease, the pharmaceutical composition including the pyrimidine derivative as an active ingredient.

Another aspect is to provide a use of the pyrimidine derivative for the manufacture of a medicament for preventing or treating BTK-mediated disease.

Another aspect is to provide a method of preventing or treating BTK-mediated disease, the method including administering the pyrimidine derivative to a subject in need thereof.

### Technical Solution

An aspect of the present disclosure provides a compound of Formula I, a solvate or stereoisomer thereof, or a pharmaceutically acceptable salt thereof:

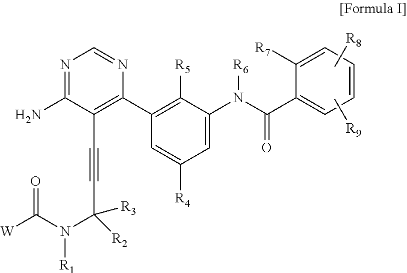

wherein, in Formula I,
W is a substituted or unsubstituted C₁-C₆ alkyl group, a substituted or unsubstituted C₂-C₆ alkenyl group, a substituted or unsubstituted C₂-C₆ alkynyl group, a substituted or unsubstituted C₁-C₆ alkoxy group, or a substituted or unsubstituted C₃-C₁₀ heterocycloalkyl group;
R₁, R₂, and R₃ are each independently hydrogen, a substituted or unsubstituted C₁-C₆ alkyl group, or a substituted or unsubstituted C₃-C₁₀ cycloalkyl group, or R₁ is linked to either R₂ or R₃ to form a 4- to 7-membered heterocyclic ring together with atoms to which they are bound;
R₆ and R₇ are each independently hydrogen, a halogen, or a substituted or unsubstituted C₁-C₆ alkyl group, or are linked to each other to form an unsaturated or partially saturated 5- to 7-membered heteroaryl ring together with atoms to which they are bound, wherein the heteroaryl ring comprises 1 to 3 hetero atoms selected from the group consisting of nitrogen (N), oxygen (O), and sulfur (S);
R₄, R₅, R₈, and R₉ are each independently hydrogen, a halogen, a cyano group, a hydroxyl group, a thiol group, a nitro group, a substituted or unsubstituted C₁-C₆ alkyl group, a substituted or unsubstituted C₂-C₆ alkenyl group, a substituted or unsubstituted C₂-C₆ alkynyl group, a substituted or unsubstituted C₁-C₆ alkoxy group, a substituted or unsubstituted C₃-C₆ cycloalkyl group, or a substituted or unsubstituted C₃-C₆ heterocycloalkyl group; and
the “substituted or unsubstituted” is being substituted with a substituent selected from a halogen, a cyano group, a hydroxyl group, a thiol group, a nitro group, a C₁-C₆ alkyl group, a C₂-C₆ alkenyl group, a C₂-C₆ alkynyl group, a C₁-C₆ alkoxy group, a C₃-C₁₀ cycloalkyl group, a C₃-C₁₀ heterocycloalkyl group, a C₃-C₁₀ aryl group, and a C₁-C₁₀ heteroaryl group or having no substituent.

Another aspect of the present disclosure provides a pharmaceutical composition for preventing or treating Bruton's tyrosine kinase (BTK)-mediated disease, the pharmaceutical composition including the compound of Formula I, the solvate or stereoisomer thereof, or the pharmaceutically acceptable salt thereof as an active ingredient, wherein the BTK-mediated disease is autoimmune disease or cancer.

Another aspect of the present disclosure provides a method of preventing or treating BTK-mediated disease, the method including administering the compound of Formula I, the solvate or stereoisomer thereof, or the pharmaceutically acceptable salt thereof to a subject, wherein the BTK-mediated disease is autoimmune disease or cancer.

### Advantageous Effects

A compound according to an aspect, a solvate or stereoisomer thereof, or a pharmaceutically acceptable salt thereof effectively inhibits the enzymatic activity of Bruton's tyrosine kinase (BTK), and thus may be useful as an active ingredient in a pharmaceutical composition for preventing or treating BTK-mediated disease such as autoimmune disease or cancer.

### MODE FOR INVENTION

Hereinafter, the present disclosure will be described in more detail.

All technical terms as used in the present specification, unless otherwise specified, have the same meaning as commonly understood by those of ordinary skill in the relevant art. In addition, suitable methods or samples are described in the present specification, but similar or equivalent ones are also within the scope of the present specification. Also, although not explicitly stated, numerical values described in the present specification are considered to include the meaning of “about”. The contents of all publications referred in the present specification are incorporated herein by reference in their entirety.

An aspect of the present disclosure provides a compound of Formula I, a solvate or stereoisomer thereof, or a pharmaceutically acceptable salt thereof:

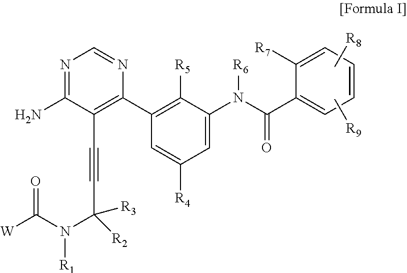

wherein, in Formula I,
W is a substituted or unsubstituted C₁-C₆ alkyl group, a substituted or unsubstituted C₂-C₆ alkenyl group, a substituted or unsubstituted C₂-C₆ alkynyl group, a substituted or unsubstituted C₁-C₆ alkoxy group, or a substituted or unsubstituted C₃-C₁₀ heterocycloalkyl group;
R₁, R₂, and R₃ are each independently hydrogen, a substituted or unsubstituted C₁-C₆ alkyl group, or a substituted or unsubstituted C₃-C₁₀ cycloalkyl group, or R₁ is linked to either R₂ or R₃ to form a 4- to 7-membered heterocyclic ring together with atoms to which they are bound;
R₆ and R₇ are each independently hydrogen, a halogen, or a substituted or unsubstituted C₁-C₆ alkyl group, or are linked to each other to form an unsaturated or partially saturated 5- to 7-membered heteroaryl ring together with atoms to which they are bound, wherein the heteroaryl ring comprises 1 to 3 hetero atoms selected from the group consisting of nitrogen (N), oxygen (O), and sulfur (S);
R₄, R₅, R₈, and R₉ are each independently hydrogen, a halogen, a cyano group, a hydroxyl group, a thiol group, a nitro group, a substituted or unsubstituted C₁-C₆ alkyl group, a substituted or unsubstituted C₂-C₆ alkenyl group, a substituted or unsubstituted C₂-C₆ alkynyl group, a substituted or unsubstituted C₁-C₆ alkoxy group, a substituted or unsubstituted C₃-C₆ cycloalkyl group, or a substituted or unsubstituted C₃-C₆ heterocycloalkyl group; and
the “substituted or unsubstituted” is being substituted with a substituent selected from a halogen, a cyano group, a hydroxyl group, a thiol group, a nitro group, a C₁-C₆ alkyl group, a C₂-C₆ alkenyl group, a C₂-C₆ alkynyl group, a C₁-C₆ alkoxy group, a C₃-C₁₀ cycloalkyl group, a C₃-C₁₀ heterocycloalkyl group, a C₃-C₁₀ aryl group, and a C₁-C₁₀ heteroaryl group or having no substituent.

The term “halogen” as used herein may be fluoro (F), chloro (Cl), bromo (Br), or iodo (I).

The term “alkyl” as used herein, unless stated otherwise, refers to a straight-chain or branched hydrocarbon residue, which may be substituted or unsubstituted. The alkyl group may include, for example, methyl, ethyl, propyl, butyl, pentyl, or all possible isomers thereof, such as isopropyl, isobutyl, and t-butyl, without limitation.

The term “alkoxy” as used herein, unless stated otherwise, refers to a straight-chain or branched hydrocarbon residue, which may be substituted or unsubstituted, linked with oxygen. The alkoxy may include, for example, methoxy, ethoxy, propoxy, and butoxy, or all possible isomers thereof, such as isopropoxy, isobutoxy, and t-butoxy, without limitation.

The term “alkenyl” as used herein refers to an unsaturated aliphatic group that is similar in length and substitutability to the above-described alkyl, but contains one or more carbon-carbon double bonds. For example, the alkenyl may include ethenyl, prophenyl, butenyl, pentenyl, hexenyl, and a branched alkenyl group, or all isomers thereof such as (E) or (Z) without limitation.

The term “alkynyl” as used herein refers to an unsaturated aliphatic group that is similar in length and substitutability to the above-described alkyl, but contains one or more carbon-carbon triple bonds. For example, the alkynyl may include ethynyl, propynyl, butynyl, pentynyl, hexynyl, and a branched alkynyl group or the like without limitation.

The term “cycloalkyl” as used herein, unless stated otherwise, refers to a saturated mono- and polycyclic hydrocarbon ring which generally have the specified number of carbon atoms including the ring, which may be substituted or unsubstituted. The cycloalkyl group may be, for example, cyclopropyl, cyclobutyl, cyclopentyl, cyclohexyl, or cycloheptyl, or the like.

The term “heterocycloalkyl” as used herein, unless otherwise stated, refers to substituted or unsubstituted cyclic alkyl including at least one, for example, 1 to 4, or 1 to 3 heteroatoms selected from the group consisting of N, O, and S, which may be monocyclic, bicyclic, tricyclic or greater, and may also be referred to as “heterocyclic ring”. Examples of monoheterocycloalkyl are piperidinyl, morpholinyl, thiomorpholinyl, pyrrolidinyl, imidazolidinyl, tetrahydrofuryl, piperazinyl, and other similar groups, but are not limited thereto. Examples of biheterocycloalkyl are diazabicyclo[2.2.1]heptyl, hexahydropyrrolo[3,4-c]pyrrolo, and other similar groups, but are not limited thereto.

The term “azacyclic ring” as used herein refers to a saturated monocyclic hydrocarbon group of the specified number of carbon atoms as defined for “cycloalkyl”, in which one carbon atom is replaced by a nitrogen atom. It may be also referred to as “azacycloalkyl” or “azahydrocarbon”. Unless otherwise stated, the azacycloalkyl refers to a cyclic aza-hydrocarbon group having: 2 to 6 ring carbon atoms and 1 nitrogen atom; 2 to 5 ring carbon atoms and 1 nitrogen atom; or 4 to 5 ring carbon atoms and 1 nitrogen atom. Examples of the azacyclic group include aziridinyl, azetidinyl, pyrrolidinyl, piperidinyl, azepinyl, dihydroazepinyl, or the like, but are not limited thereto.

The term “aryl” as used herein, unless stated otherwise, refers to an aromatic group that may be substituted or unsubstituted, and may include, for example, C₆-C₃₀ aryl, C₆-C₂₀ aryl, or C₆-C₁₀ aryl, in which double bonds alternate (resonate) between adjacent carbon atoms or suitable heteroatoms. For example, the aryl may include phenyl, biphenyl, naphtyl, toluyl, naphthalenyl, anthracenyl, or any possible isomers thereof without limitation.

The term “heteroaryl” as used herein, unless stated otherwise, refers to monocyclic or bicyclic or greater, substituted or unsubstituted aromatic group including at least one, for example, 1 to 4, or 1 to 3 heteroatoms selected from the group consisting of N, O, and S, and may also be referred to as “heteroaryl ring”. Examples of the monocyclic heteroaryl are thiazolyl, oxazolyl, thiophenyl, furanyl, pyrrolyl, imidazolyl, isoxazolyl, pyrazolyl, triazolyl, thiadiazolyl, tetrazolyl, oxadiazolyl, pyridinyl, pyridazinyl, pyrimidinyl, pyrazinyl, and other similar groups, but are not limited thereto. Examples of the bicyclic heteroaryl are indolyl, benzothiophenyl, benzofuranyl, benzimidazolyl, benzoxazolyl, benzisoxazolyl, benzthiazolyl, benzthiadiazolyl, benztriazolyl, quinolinyl, isoquinolinyl, oxoisoquinolinyl, purinyl, furopyridinyl, oxochromen, dioxoisoindoline, pyrazolopyridinyl, pyrazolo[1,5-a]pyridinyl, and other similar groups, but are not limited thereto.

The term “partially saturated” as used herein refers to inclusion of at least one saturated site, i.e., at least one single bond, within the aryl or heteroaryl ring as defined above. The term “unsaturated” as used herein refers to no inclusion of a saturated site, i.e., a single bond, within the aryl or heteroaryl ring as defined above.

The term “substituted” as used herein, unless stated otherwise refers that at least one hydrogen atom is replaced by a non-hydrogen group, and for example, refers to a substitution with a substituent selected from a halogen, a cyano group, a hydroxyl group, a thiol group, a nitro group, a C₁-C₆ alkyl group, a C₂-C₆ alkenyl group, a C₂-C₆ alkynyl group, a C₁-C₆ alkoxy group, a C₃-C₁₀ cycloalkyl group, a C₃-C₁₀ heterocycloalkyl group, a C₃-C₁₀ aryl group, and a C₁-C₁₀ heteroaryl group.

A numerical range indicated with the term “to” in the present specification refers to a range including numerical values described before and after the term “to” as the lower limit and the upper limit, respectively.

The term “solvate” as used herein may include a molecular composite including a compound and one or more pharmaceutically acceptable solvent molecules such as ethanol or water. A composite in which the solvent molecule is water is also referred to as “hydrate”.

The term “stereoisomer” as used herein refers to the compound of the present disclosure or the salt thereof having the same chemical or molecular formula, but being optically or sterically different, and may specifically include a diastereoisomer, an enantiomer, a geometric isomer, or a conformational isomer.

The term “derivative” as used herein refers to a compound obtained by substituting a part of the structure of the compound with a different atom or atomic group.

In addition, the compound may be used in the form of a pharmaceutically acceptable salt derived from an inorganic acid or an organic acid, and for example, the salt may be a salt derived from hydrochloric acid, hydrobromic acid, sulfuric acid, phosphoric acid, nitric acid, acetic acid, glycolic acid, lactic acid, pyruvic acid, malonic acid, succinic acid, glutaric acid, fumaric acid, malic acid, mandelic acid, tartaric acid, citric acid, ascorbic acid, palmitic acid, maleic acid, hydroxymaleic acid, benzoic acid, hydroxybenzoic acid, phenylacetic acid, cinnamic acid, salicyclic acid, methanesulfonic acid, benzenesulfonic acid, or toluenesulfonic acid, or the like.

The pharmaceutically acceptable salt of the compound may be prepared by dissolving the compound of Formula I in a water-miscible organic solvent, such as acetone, methanol, ethanol, or acetonitrile, and adding an excess of organic acid or an aqueous acid solution of an inorganic acid, followed by precipitating or recrystallizing it. Subsequently, the solvent or an excess of acid is evaporated from the mixture and dried to obtain an addition salt, or the precipitated salt may be filtered by suction to prepare the pharmaceutically acceptable salt.

In an embodiment, W may be a substituted or unsubstituted C₂-C₆ alkenyl group; R₁, R₂, and R₃ may each independently be hydrogen or a substituted or unsubstituted C₁-C₆ alkyl group, or R₁ may be linked to either R₂ or R₃ to form a 4- to 7-membered heterocyclic ring together with atoms to which they are bound; R₆ and R₇ may each independently be hydrogen, a halogen, or a substituted or unsubstituted C₁-C₆ alkyl group, or may be linked to each other to form an unsaturated or partially saturated 6-membered heteroaryl ring together with atoms to which they are bound, wherein the heteroaryl ring may include 1 to 3 nitrogen atoms; and R₄, R₅, R₈, and R₉ may each independently be hydrogen, a halogen, a substituted or unsubstituted C₁-C₆ alkyl group, or a substituted or unsubstituted C₃-C₆ cycloalkyl group.

In an embodiment, W may be a substituted or unsubstituted C₂-C₆ alkenyl group; R₁, R₂, and R₃ may each independently be hydrogen or a substituted or unsubstituted C₁-C₃ alkyl group, or R₁ may be linked to either R₂ or R₃ to form a 4- to 7-membered azacyclic ring together with atoms to which they are bound; R₄ and R₅ may each independently be hydrogen, a halogen, or a substituted or unsubstituted C₁-C₃ alkyl group; R₆ and R₇ may each independently be hydrogen, a halogen, or a substituted or unsubstituted C₁-C₃ alkyl group, or may be linked to each other to form an unsaturated or partially saturated 6-membered heteroaryl ring together with atoms to which they are bound, wherein the heteroaryl ring may include 1 to 3 nitrogen atoms; R₈ may be hydrogen, a halogen, a substituted or unsubstituted C₁-C₆ alkyl group, or a substituted or unsubstituted C₃-C₆ cycloalkyl group; and R₉ may be hydrogen, a halogen, or a substituted or unsubstituted C₁-C₃ alkyl group.

In an embodiment, R₈ and R₉ may be present in a meta-position with respect to R₇. For example, the compound of Formula I may have the same positional relationship of R₈ and R₉ as in Formula I-1 below:

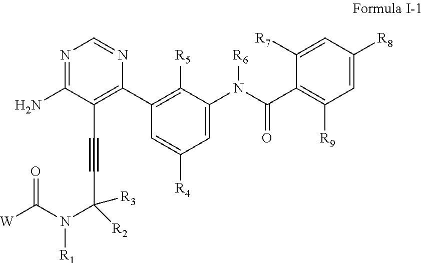

wherein, in Formula I-1,
W, R₁, R₂, R₃, R₄, R₅, R₆, R₇, R₈, and R₉ are as defined in Formula I.

In an embodiment, W may be

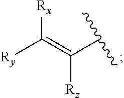

Rₓ, R_{y}, and R_{z} may each independently be hydrogen, a halogen, a substituted or unsubstituted C₁-C₆ alkyl group, a cyano group, or —(CH₂)ₙ—NR₁₀R₁₁; n may be an integer from 0 to 6; and R₁₀ and R₁₁ may each independently be a substituted or unsubstituted C₁-C₆ alkyl group, or may be linked to each other to form a 4- to 7-membered azacyclic ring together with nitrogen (N) atoms to which they are bound.

In an embodiment, W may be

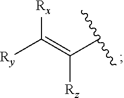

Rₓ may be hydrogen, a halogen, or a substituted or unsubstituted C₁-C₃ alkyl group; R_{y} may be hydrogen, a halogen, a substituted or unsubstituted C₁-C₃ alkyl group, or —(CH₂)ₙ—NR₁₀R₁₁; R_{z} may be hydrogen, a halogen, a substituted or unsubstituted C₁-C₆ alkyl group, or a cyano group; n may be an integer of 0 or 1; and R₁₀ and R₁₁ may each independently a substituted or unsubstituted C₁-C₃ alkyl group, or may be linked to each other to form a 4- to 7-membered azacyclic ring together with nitrogen (N) atom to which they are bound.

In an embodiment, the compound of Formula I may be a compound of Formula Ia:

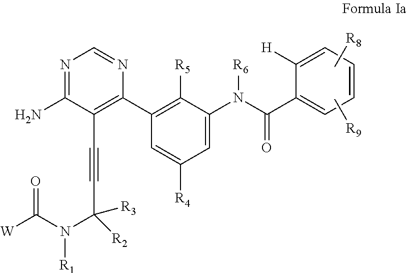

wherein, in Formula Ia,
W, R₁, R₂, R₃, R₄, R₅, R₆, R₈, and R₉ are as defined in Formula I.

In an embodiment, the compound of Formula I may be a compound of Formula Ib:

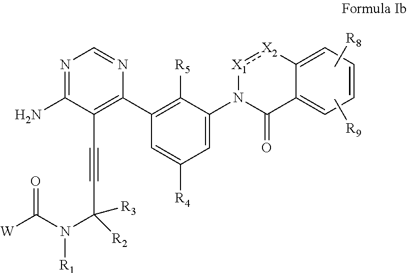

wherein, in Formula Ib, [symbol]
X₁ and X₂ may each independently be —C(R₁₂)₂—, —CR₁₂—, or —N—; [symbol] ′ is a single bond or a double bond; R₁₂ may be hydrogen or a substituted or unsubstituted C₁-C₆ alkyl group; and W, R₁, R₂, R₃, R₄, R₅, R₈, and R₉ are as defined in Formula I.

In an embodiment, the compound of Formula I may be selected from the group consisting of Compounds 1) to 23):
1) N-(3-(6-amino-5-(3-N-methylacrylamido)prop-1-yn-1-yl)pyrimidin-4-yl)5-fluoro-2-methylphenyl)-2-fluoro-4-isopropylbenzamide;
2) N-(3-(6-amino-5-(3-N-methylacrylamido)prop-1-yn-1-yl)pyrimidin-4-yl)5-fluoro-2-methylphenyl)-4-cyclopropyl-2-fluorobenzamide;
3) N-(3-(4-amino-6-(3-(6-cyclopropyl-8-fluoro-1-oxo-3,4-dihydroisoquinolin-2(1H)-yl)-5-fluorophenyl)pyrimidin-5-yl)prop-2-yn-1-yl)-N-methylacrylamide;
4) N-(3-(4-amino-6-(3-(6-cyclopropyl-8-fluoro-1-oxo-3,4-dihydroisoquinolin-2(1H)-yl)-5-fluoro-2-methylphenyl)pyrimidin-5-yl)prop-2-yn-1-yl)-N-methylacrylamide;
5) N-(3-(4-amino-6-(3-(6-cyclo-8-fluoro-1-oxoisoquinolin-2(1H)-yl)-5-fluorophenyl)pyrimidin-5-yl)prop-2-yn-1-yl)-N-methylacrylamide;
6) N-(3-(4-amino-6-(3-(6-cyclopropyl-8-fluoro-1-oxoisoquinolin-2(1H)-yl)-5-fluoro-2-methylphenyl)pyrimidin-5-yl)prop-2-yn-1-yl)-N-methylacrylamide;
7) N-(3-(4-amino-6-(3-(6-cyclopropyl-8-fluoro-1-oxoisoquinolin-2(1H)-yl)-2,5-difluorophenyl)pyrimidin-5-yl)prop-2-yn-1-yl)-N-methylacrylamide;
8) N-(3-(4-amino-6-(3-(6-cyclopropyl-8-fluoro-1-oxoisoquinolin-2(1H)-yl)-2,5-dimethylphenyl)pyrimidin-5-yl)prop-2-yn-1-yl)-N-methylacrylamide;
9) N-(3-(4-amino-6-(3-(6-cyclopropyl-8-fluoro-1-oxoisoquinolin-2(1H)-yl)-5-fluoro-2-methylphenyl)pyrimidin-5-yl)prop-2-yn-1-yl)acrylamide;
10) N-(3-(4-amino-6-(3-(6-(tert-butyl)-8-fluoro-1-oxophthalazin-2-(1H)-yl)-5-fluorophenyl)pyrimidin-5-yl)prop-2-yn-1-yl)-N-methylacrylamide;
11) N-(3-(4-amino-6-(3-(6-cyclopropyl-1-oxophthalazin-2(1H)-yl)-5-fluoro-2-methylphenyl)pyrimidin-5-yl)prop-2-yn-1-yl)-N-methylacrylamide;
12) N-(3-(4-amino-6-(3-(6-cyclopropyl-8-fluoro-1-oxophthalazin-2(1H)-yl)-5-fluoro-2-methylphenyl)pyrimidin-5-yl)prop-2-yn-1-yl)-N-methylacrylamide;
13) N-(3-(4-amino-6-(3-(6-(tert-butyl)-8-fluoro-1-oxophthalazin-2(1H)-yl)-5-fluoro-2-methylphenyl)pyrimidin-5-yl)prop-2-yn-1-yl)-N-methylacrylamide;
14) N-(3-(4-amino-6-(3-(6-tert-butyl)-1-oxophthalazin-2-(1H)-yl)-5-fluorophenyl)pyrimidin-5-yl)prop-2-yn-1-yl)-N-methylacrylamide;
15) N-(3-(4-amino-6-(3-(7-cyclopropyl-5-fluoro-4-oxoquinazolin-3(4H)-yl)-5-fluorophenyl)pyrimidin-5-yl)prop-2-yn-1-yl)-N-methylacrylamide;
16) (R)-2-(3-(5-((1-acryloylpyrrolidin-2-yl)ethynyl)-6-aminopyrimidin-4-yl)-5-fluoro-2-methylphenyl)-6-cyclopropyl-8-fluoroisoquinolin-1(2H)-one;
17) (S)-2-(3-(5-((1-acryloylpyrrolidin-2-yl)ethynyl)-6-aminopyrimidin-4-yl)-5-fluoro-2-methylphenyl)-6-cyclopropyl-8-fluoroisoquinolin-1(2H)-one;
18) (S)-2-(3-(5-((1-acryloylpyrrolidin-2-yl)ethynyl)-6-aminopyrimidin-4-yl)-5-fluoro-2-methylphenyl)-8-fluoro-6-isoquinolin-1-(2H)-one;
19) (S)-2-(3-(6-amino-5-((1-(2-fluoroacryloyl)pyrrolidin-2-yl)ethynyl)pyrimidin-4-yl)-5-fluoro-2-methylphenyl)-6-cyclopropyl-8-fluoroisoquinolin-1(2H)-one;
20) (S)-2-(3-(6-amino-5-((1-(2-chloroacryloyl)pyrrolidin-2-yl)ethynyl)pyrimidin-4-yl)-5-fluoro-2-methylphenyl)-6-cyclopropyl-8-fluoroisoquinolin-1(2H)-one;
21) (S)—N-(3-(5-((1-acryloylpyrrolidin-2-yl)ethynyl)-6-aminopyrimidin-4-yl)-5-fluoro-2-methylphenyl)-4-cyclopropyl-2-fluorobenzamide;
22) 2-(3-(5-((1-acryloylpiperidin-2-yl)ethynyl)-6-aminopyrimidin-4-yl)-5-fluoro-2-methylphenyl)-6-cyclopropyl-8-isoquinolin-1(2H)-one; and
23) (S)-2-(3-(5-((1-acryloylpiperidin-2-yl)ethynyl)-6-aminopyrimidin-4-yl)-5-fluoro-2-methylphenyl)-8-fluoro-6-isopropylisoquinolin-1(2H)-one.

The compound according to an aspect having a triple bond bonded to the amino pyrimidine backbone may effectively inhibit the BTK activity.

Another aspect provides a pharmaceutical composition for preventing or treating Bruton's tyrosine kinase (BTK)-mediated disease, the pharmaceutical composition including the compound of Formula I, the solvate or stereoisomer thereof, or the pharmaceutically acceptable salt thereof as an active ingredient, wherein the BTK-mediated disease is autoimmune disease or cancer.

The term “treating” or “treatment” as used herein refers to: inhibition of a disease, for example, inhibition of a disease, pathologic condition, or disorder in a subject experiencing or exhibiting a pathology or symptom of a disease, pathologic condition, or disorder, i.e., preventing further development of pathology and/or symptoms; or ameliorating a disease, for example, ameliorating a disease, pathologic condition, or disorder in a subject or experiencing or exhibiting a pathology or symptom of the disease, pathologic condition, or disorder, i.e., reversing a pathology and/or symptom, for example, reducing severity of the disease.

The term “preventing” or “prevention” as used herein refers to preventing a disease, for example, preventing a disease, pathologic condition, or disorder in a subject who is predisposed to a disease, pathologic condition, or disorder, but has not yet experienced or exhibited a pathology or symptom of the disease.

The term “subject” or “patient” as used herein refers to any animals including mammals, for example, mice, rats, other rodents, rabbits, dogs, cats, pigs, cows, sheep, horses, or primates, and humans.

The term “BTK-mediated disease” refers to a disease that occurs due to excessive signaling upon overactivity of BTK, and may refer to a condition or disease in which prevention, amelioration, or treatment effects can be expected by inhibiting the BTK activity. The BTK-mediated disease may include an autoimmune disease or cancer. The term “autoimmune disease” as used herein refers to any group of diseases in which tissue damage is associated with a humoral or cell-mediated response to the components of the body itself. The autoimmune diseases may include, for example, rheumatic arthritis, psoriasis arthritis, osteoarthritis, Still's disease, juvenile arthritis, lupus, myasthenia gravis, Hashimoto's thyroiditis, iodide thyroiditis, Graves' disease, Sjogren's syndrome, multiple sclerosis, Guillain-Barre syndrome, acute disseminated encephalomyelitis, Addison's disease, nystagmus-myoclonus syndrome, ankylosing spondylitis, antiphospholipid antibody syndrome, aplastic anemia, autoimmune hepatitis, celiac disease, Goodpasture's syndrome, idiopathic thrombocytopenic purpura, optic neuritis, scleroderma, primary biliary cirrhosis, Reiter's syndrome, Takayasu arteritis, temporal arteritis, warm autoimmune hemolytic anemia, Wegener's granulomatosis, psoriasis, alopecia universalis, Behcet's disease, autonomic dysfunction, asthma, chronic spontaneous urticaria, pemphigus, systemic lupus erythematosus, hidradenitis, skin disease, immunoglobulin G4-related disease, endometrioma, interstitial cystitis, neuromyotonia, or vulvodynia, but are not limited thereto.

The term “cancer” as used herein refers to a physiological condition in mammals that is typically characterized by uncontrolled cell growth. In the present specification, the cancer may include carcinoma, lymphoma, blastoma, sarcoma, leukemia, or lymphoid malignancy. More specific examples of the cancer are squamous cell cancer (e.g., epithelial squamous cell cancer), lung cancer, such as small-cell lung cancer, non-small cell lung cancer (“NSCLC”), adenocarcinoma of the lung, and squamous carcinoma of the lung, cancer of the peritoneum, hepatocellular cancer, gastric or stomach cancer, such as gastrointestinal cancer, pancreatic cancer, glioblastoma, cervical cancer, ovarian cancer, liver cancer, bladder cancer, hepatoma, breast cancer, colon cancer, rectal cancer, large intestine cancer, endometrial or uterine carcinoma, salivary gland carcinoma, kidney or renal cancer, prostate cancer, vulval cancer, thyroid cancer, hepatic carcinoma, anal carcinoma, penile carcinoma, and head and neck cancer.

In addition, the cancer in the present disclosure may be B-cell malignancy. The B-cell malignancy may be, for example, any disease selected from the group consisting of chronic lymphocytic leukemia (CLL), mantle cell lymphoma (MCL), B-cell prolymphocytic leukemia, diffuse large B cell lymphoma (DLBCL), splenic marginal zone lymphoma (SMZL), Burkitt's lymphoma, B-cell non-Hodgkin's lymphoma, follicular lymphoma, primary central nervous system lymphoma (PCNSL), nodular lymphocyte predominant Hodgkin's lymphoma (NLPHL), hairy cell leukemia, splenic lymphoma, lymphoplasmacytic lymphoma, extranodal marginal zone lymphoma of the mucosa-associated lymphoid tissue (MALT lymphoma), nodal marginal zone lymphoma, pediatric nodal marginal zone lymphoma, primary cutaneous follicle-center lymphoma, T-cell/histiocyte-rich large B-cell lymphoma, lymphomatoid granulomatosis, primary mediastinal (thymic) large B-cell lymphoma, intravascular large B-cell lymphoma, ALK-positive large B-cell lymphoma, plasmablastic lymphoma, and primary effusion lymphoma. In an embodiment, the pharmaceutical composition including the compound of Formula I, the solvate or stereoisomer thereof, or the pharmaceutically acceptable salt thereof as an active ingredient exhibits inhibitory activity against BTK enzymes, and thus may be used to treat BTK-mediated disease such as autoimmune disease or cancer.

In an embodiment, the pharmaceutical composition may include a conventional pharmaceutically acceptable carrier, excipient, or additive. The pharmaceutical composition may be formulated according to a conventional method, and may be prepared in various oral dosage forms such as tablets, pills, powders, capsules, syrups, emulsions, microemulsions, or the like, or in parenteral dosage forms such as intramuscular, intravenous, or subcutaneous administration.

When the pharmaceutical composition is prepared in the form of an oral dosage form, examples of additives or carriers used are cellulose, calcium silicate, corn starch, lactose, sucrose, dextrose, calcium phosphate, stearic acid, magnesium stearate, calcium stearate, gelatin, talc, a surfactant, a suspending agent, an emulsifier, a diluent, and the like. When the pharmaceutical composition is prepared in the form of an injection, the additive or carrier may include water, saline, glucose aqueous solution, similar sugar aqueous solution, alcohol, glycol, ether (e.g., polyethylene glycol 400), oil, fatty acid, fatty acid ester, glyceride, a surfactant, a suspending agent, an emulsifier, or the like.

A dose of the pharmaceutical composition is an amount effective for the treatment or prevention of a subject or patient, and may be administered orally or parenterally depending on the purpose. It may be administered in one to several divided doses to provide a dose of 0.01 mg to 1,000 mg, for example, 0.1 mg to 300 mg, per kg of body weight per day, based on the active ingredient, when administered orally, and a dose of 0.01 mg to 100 mg, for example, 0.1 mg to 50 mg, per kg of body weight per day, based on the active ingredient, when administered parenterally. The administration dose for a specific subject or patient is to be determined depending on several related factors, such as the body weight, age, gender, health conditions, and diet of patients, an administration period, an administration method, severity of diseases, and the like. It is to be understood that the administration dose may be appropriately increased or decreased by experts, and the administration dose is not intended to limit the scope of the present disclosure in any way. A physician or veterinarian having ordinary skill in the relevant art can easily determine and prescribe an effective amount of a required pharmaceutical composition. For example, the administration dose may start the dose of compounds of the present disclosure used in a pharmaceutical composition at a lower level than required to achieve a desired therapeutic effect, and gradually increase the dose until the desired effect is achieved.

In an embodiment, the pharmaceutical composition includes within its scope a pharmaceutical composition comprising, as an active ingredient, a therapeutically effective amount of at least one of the compounds according to an embodiment, alone or in combination with a pharmaceutical carrier. For example, the compound according to an embodiment may be administered alone, in combination with a compound according to another embodiment, or in combination with one or more other therapeutic agents or other pharmaceutically active substances simultaneously, separately, or sequentially. Also, as known in the art, other combination therapies including a BTK inhibitor are also within the scope of the present disclosure.

Another aspect provides a method of preventing or treating BTK-mediated disease, the method including administering the compound of Formula I, the solvate or stereoisomer thereof, or the pharmaceutically acceptable salt thereof, or the pharmaceutical composition including any of the foregoing, to a subject in need thereof, wherein the BTK-mediated disease is autoimmune disease or cancer.

Details of the method of the prevention or treatment may be directly applied by the description of the pharmaceutical composition according to an aspect of the present disclosure.

Also, doses used in the method of the prevention or treatment may be effective amounts for the treatment or prevention in a subject or patient, and the description of the dose of the pharmaceutical composition may be directly applied.

Another aspect provides medical use of the compound of Formula I, the solvate or stereoisomer thereof, or the pharmaceutically acceptable salt thereof for the manufacture of a medicament for preventing or treating BTK-mediated disease, wherein the BTK-mediated disease is autoimmune disease or cancer.

Details of the medical use may be directly applied by the description of the pharmaceutical composition according to an aspect of the present disclosure.

Hereinafter, a preparation method for the compound of Formula I will be described in detail.

The compound of Formula I may be typically prepared according to the method shown in Reaction Scheme 1, but is not limited thereto. Those skilled in the art in the field of organic compounds may appropriately adjust specific reaction pathways, reaction conditions, reaction amounts, etc., using different synthetic methods so that the compound may be prepared by methods other than those specifically described in the following embodiments.

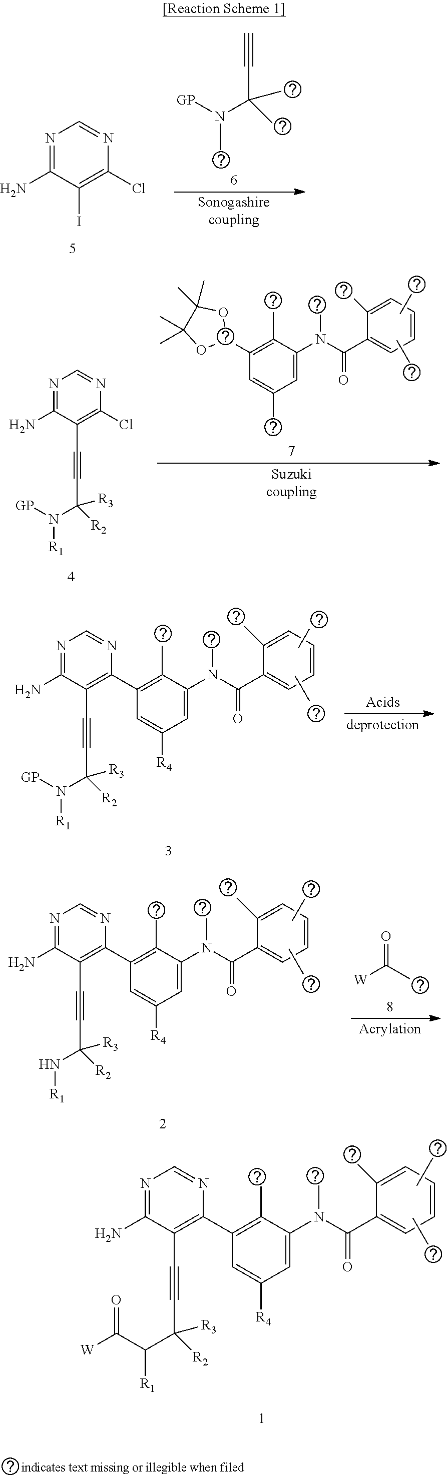

Regarding more detail with reference to Reaction Scheme 1 above,
the compound of Formula 5 and the compound of Formula 7 may be prepared from commercially available compounds by using common knowledge in the field of organic chemistry.

In Reaction Scheme 1, R₁, R₂, R₃, R₄, R₅, R₆, R₇, R₈, R₉, and W are as defined in Formula I.

The compound of Formula 5 and the compound of Formula 6 may be subjected to Sonogashira coupling using an appropriate palladium catalyst, such as bis(triphenylphosphine)palladium (II) dichloride, so as to prepare the compound of Formula 4. Here, the solvent used in the reaction may be any solvent that does not inhibit the reaction. The solvent used in the reaction may be: a polar aprotic solvent such as dimethyl sulfoxide, N,N-dimethylformamide, acetonitrile, tetrahydrofuran, or the like; a polar protic solvent such as methanol, ethanol, 2-propanol, 2-isopropanol, 2-butanol, or the like; or a non-polar aprotic solvent such as toluene, 1,4-dioxane, or the like. In detail, the solvent may be N,N-dimethyl formamide. A temperature for the reaction may be 0° C. to 150° C., and may preferably be 100° C.

The compound of Formula 4 and the boronic acid ester compound of Formula 7 may be subjected to Suzuki coupling, so as to prepare the compound of Formula 3. Here, the solvent used in the reaction may be diethylene glycol dimethyl ether:water (7:1), and a preferable temperature for the reaction may be 100° C.

The compound of Formula 3 may react with an organic acid, such as trifluoroacetic acid, or an inorganic acid, such as concentrated hydrochloric acid, in an organic solvent, such as dichloromethane, to deprotect the t-butoxycarbonyl group, thereby preparing the compound of Formula 2. A temperature for the reaction may be 0° C. to room temperature, and may preferably be room temperature.

The compound of Formula 2 may react with various acids using an appropriate coupling reagent, such as T3P, and an appropriate base, such as N,N-diisopropylamine, so as to prepare the compound of Formula 1. Here, the solvent used in the reaction may be any solvent that does not inhibit the reaction. The solvent used in the reaction may be: a polar aprotic solvent such as dimethyl sulfoxide, N,N-dimethylformamide, acetonitrile, tetrahydrofuran, or the like; a polar protic solvent such as methanol, ethanol, 2-propanol, 2-isopropanol, 2-butanol, or the like; or a non-polar aprotic solvent such as toluene, 1,4-dioxane, or the like. In detail, the solvent may be N,N-dimethyl formamide. A temperature for the reaction may be 0° C. to room temperature, and may preferably be 0° C.

The pharmaceutical composition including the compound of Formula I synthesized by the aforementioned preparation method, the solvate or stereoisomer thereof, or the pharmaceutically acceptable salt thereof as an active ingredient may effectively inhibit enzymatic activity of BTK, and thus may be used to treat BTK-mediated disease such as autoimmune disease or cancer.

Hereinafter, the present disclosure will be described in detail by Examples below. However, Examples below are for illustrating the present disclosure, and the contents of the present disclosure are not limited thereto.

### EXAMPLES

### Example 1: Preparation of N-(3-(6-amino-5-(3-N-methylacrylamido)prop-1-yn-1-yl)pyrimidin-4-yl)5-fluoro-2-methylphenyl)-2-fluoro-4-isopropylbenzamide

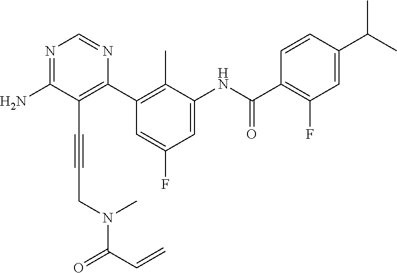 [SMILES: C=CC(=O)N(C)CC#Cc1c(N)ncnc1-c1cc(F)cc(NC(=O)c2ccc(C(C)C)cc2F)c1C]

### Step 1) Preparation of methyl 2-fluoro-4-isopropylphenyl-benzoate

10.0 g (42.9 mmol) of methyl 4-bromo-2-fluoro-benzoate, 2-isoprophenylboronic acid, 12.5 mL (64.4 mmol) of pinacol ester, 3.6 g (4.3 mmol) of palladium(II) dichloride dichloromethane complex, and 17.9 g (128.7 mmol) of calcium carbonate were diluted with 200 mL of 1,4-dioxane:water (4:1), and the reaction mixture was stirred at 80° C. for 4 hours. After completion of the reaction, the reaction product was cooled to room temperature, diluted with ethyl acetate, and washed with saturated brine. The organic layer thus separated was dried with anhydrous sodium sulfate and filtered under reduced pressure, and then the residues obtained by distillation under reduced pressure were separated by column chromatography (ethyl acetate:hexane at a volume ratio of 1:50), so as to obtain 8.17 g (yield: 98%) of the title compound.

¹H-NMR (300 MHz, CDCl₃): δ 7.90˜7.84 (t, 1H), 7.28˜7.24 (m, 1H), 7.20˜7.16 (m, 1H), 5.47 (s, 1H), 5.21 (s, 1H), 3.91 (s, 3H), 2.12 (s, 3H).

### Step 2) Preparation of methyl 2-fluoro-4-isopropylbenzoate

8.17 g (42.1 mmol) of the compound prepared in step 1) was dissolved in 140 mL of methanol, and 2.0 g (8.4 mmol) of platinum (IV) oxide was added thereto. The reaction mixture was degassed and stirred under hydrogen for 10 minutes, and then stirred again under hydrogen for 18 hours at 25° C. After completion of the reaction, the reaction product was filtered through a celite-filled filter under reduced pressure, and the filtrate was distilled under reduced pressure, so as to obtain 8.16 g (yield: 99%) of the title compound.

¹H-NMR (300 MHz, DMSO-d₆): δ 7.82˜7.76 (m, 1H), 7.23˜7.17 (m, 2H), 3.82 (s, 3H), 3.01˜2.88 (s, 1H), 1.20˜1.17 (d, 6H).

### Step 3) Preparation of 2-fluoro-4-isopropylbenzoic acid

8.16 g (41.6 mmol) of the compound prepared in step 2) was diluted with 100 mL of tetrahydrofuran:methanol:water (3:3:1) and cooled to 0° C. 5.5 g (124.8 mmol) of lithium hydroxide monohydrate was added thereto, and the resulting reaction mixture was stirred at 25° C. for 4 hours. After completion of the reaction, the reaction product was distilled under reduced pressure. The residues thus obtained were acidified with 1 N-hydrochloric acid (pH 3 to 4) to produce a solid. The solid was filtered, and the filtrate was washed twice with 100 mL of water. The resulting solid was dried in a drying oven at 50° C. to obtain 6.1 g (yield: 81%) of the title compound.

¹H-NMR (300 MHz, DMSO-d₆): δ 13.02 (brs, 1H), 7.80˜7.75 (m, 1H) 7.18˜7.14 (m, 2H), 2.98˜2.87 (m, 1H), 1.20˜1.18 (d, 6H).

### Step 4) Preparation of 2-(5-fluoro-2-methyl-3-nitrophenyl)-4,4,5,5-tetramethyl-1,3,2-dioxaborolane

30 g (124.4 mmol) of 2-bromo-4-fluoro-6-nitrotoluene was dissolved in 1.0 L of 1,4-dioxane, and 48.3 g (186.5 mmol) of bis(pinacolato)diboron, 44 g (435.2 mmol) of acetic acid potassium, and 5.2 g (6.2 mmol) of palladium (II) dichloride dichloromethane complex were added thereto. The reaction mixture was stirred under reflux at 100° C. for 2 hours. After completion of the reaction, the reaction product was cooled to room temperature and filtered through a celite-filled filter under reduced pressure, and the filtrate was distilled under reduced pressure. The residues thus obtained were separated by column chromatography (ethyl acetate:hexane at a volume ratio of 1:50), so as to obtain 34.2 g (yield: 98%) of the title compound.

¹H-NMR (300 MHz, CDCl₃): δ 7.67˜7.64 (m, 1H), 7.54˜7.50 (m, 1H), 2.61 (s, 3H), 1.33 (s, 12H).

### Step 5) Preparation of 5-fluoro-2-methyl-3-(4,4,5,5-tetramethyl-1,3,2-dioxaborolan-2-yl)aniline

34.1 g (121.3 mmol) of the compound obtained in step 4) was diluted with 340 mL of ethyl acetate, and 3.4 g (30.4 mmol) of 10% palladium immersed on activated carbon was added thereto. The reaction mixture was degassed and stirred under hydrogen for 10 minutes, and then stirred again under hydrogen for 18 hours at 25° C. After completion of the reaction, the reaction product was filtered through a celite-filled filter under reduced pressure, and the filtrate was distilled under reduced pressure, so as to obtain 28.7 g (yield: 94%) of the title compound.

¹H-NMR (300 MHz, CDCl₃): δ 6.90˜6.87 (m, 1H), 6.48˜6.45 (m, 1H), 3.69 (brs, 2H), 2.32 (s, 3H), 1.35 (s, 12H).

### Step 6) Preparation of 2-fluoro-N-(5-fluoro-2-methyl-3-(4,4,5,5-tetramethyl-1,3,2-dioxaborolan-2-yl)phenyl)-4-isopropylbenzamide

7.89 g (31.4 mmol) of the compound obtained in step 5) and 5.72 g (31.4 mmol) of the compound obtained in step 3) were diluted with 100 mL of N,N-dimethyl formamide, and 24.4 g (62.8 mmol) of 1-[bis(dimethylamino)methylene]-1H-1,2,3-triazolo[4,5-b]pyridium 3-oxide hexafluorophosphate and 16.6 mL (94.3 mmol) of N,N-diisopropyl ethyleneamine were added thereto. The reaction mixture was stirred at 50° C. for 14 hours. After completion of the reaction, the reaction product was cooled to room temperature, diluted with ethyl acetate, and washed with distilled water. The organic layer thus separated was dried with anhydrous sodium sulfate and filtered under reduced pressure, and then the residues obtained by distillation under reduced pressure were separated by column chromatography (ethyl acetate:hexane at a volume ratio of 1:20), so as to obtain 8.3 g (yield: 64%) of the title compound.

### Step 7) Preparation of 6-chloro-5-iodo-pyrimidine-4-amine

60 g (463.1 mmol) of 6-chloropyrimidine-4-amine was diluted with 360 mL of N,N-dimethyl formamide, and 108.6 g (463.1 mmol) of N-iodosuccinimide was added thereto. The reaction mixture was stirred at 100° C. for 4 hours. The reaction mixture was cooled to room temperature, diluted with ethyl acetate, and washed with sodium thiosulfate aqueous solution. The solid thus obtained was filtered, and the filtrate was washed twice with 500 mL of distilled water. The resulting solid was dried in a drying oven at 50° C. to obtain 48.8 g (yield: 41%) of the title compound.

### Step 8) Preparation of tert-butyl methyl(prop-2-yn-1-yl)carbamate

50 g (322.2 mmol) of tert-butyl prop-2-yn-1-yl carbamate was diluted with 500 mL of N,N-dimethyl formamide, and then cooled to 0° C. 14.2 g (354.4 mmol) of sodium hydride was added in several portions at 0° C., and the reaction mixture was stirred for 30 minutes. 26.4 mL (418.8 mmol) of iodine (1) was added thereto, and the resulting reaction mixture was stirred at 50° C. for 3 hours. After completion of the reaction, the reaction product was cooled to room temperature, diluted with ethyl acetate, and washed with distilled water. The organic layer thus separated was dried with anhydrous sodium sulfate and filtered under reduced pressure, and the filtrate was distilled under reduced pressure, so as to obtain 49.2 g (yield: 90%) of the title compound.

¹H-NMR (300 MHz, CDCl₃): δ 4.03 (bs, 2H), 2.90 (s, 3H), 2.21 (s, 1H), 1.46 (s, 9H).

### Step 9) Preparation of tert-butyl (3-(4-amino-6-chloropyrimidin-5-yl)prop-2-yn-1-yl)(methyl)carbamate

17.8 g (69.7 mmol) of the compound prepared in step 7) was diluted with 90 mL of N,N-dimethyl formamide, and 2.5 g (3.5 mmol) of bis(triphenylphosphine)palladium (II) dichloride and 677 mg (3.5 mmol) of copper iodide were added thereto under nitrogen. 17.7 g (104.5 mmol) of tert-butyl methyl(prop-2-yn-1-yl)carbamate solution diluted with 45 mL of N,N-dimethyl formamide and 19.6 mL (139.4 mmol) of triethylamine solution diluted with 45 mL of N,N-dimethyl formamide were added thereto at 25° C., and the resulting reaction mixture was stirred under nitrogen at 35° C. for 18 hours. After completion of the reaction, the reaction product was cooled to room temperature, diluted with ethyl acetate, and washed with distilled water and sodium bicarbonate aqueous solution. The organic layer thus separated was dried with anhydrous sodium sulfate and filtered under reduced pressure, and then the residues obtained by distillation under reduced pressure were separated by column chromatography (ethyl acetate:hexane at a volume ratio of 1:5), so as to obtain 7.1 g (yield: 34%) of the title compound.

¹H-NMR (300 MHz, DMSO-d₆): δ 8.17 (s, 1H), 4.30 (bs, 2H), 2.87 (s, 3H), 1.40 (s, 9H).

### Step 10) Preparation of tert-butyl (3-(4-amino-6-(5-fluoro-3-(2-fluoro-4-isopropylbenzamido)-2-methylphenyl)pyrimidin-5-yl)prop-2-yn-1-yl)(methyl)carbamate

1 g (3.4 mmol) of the compound of step 9) and 1.7 g (4.0 mmol) of the compound of step 6) were diluted with 40 mL of dimethoxyethane:distilled water (at a volume ratio of 7:1), and 10.2 mL (10.2 mmol) of 1 M-sodium hydrogen carbonate was added thereto. The reaction mixture was degassed and stirred under nitrogen for 10 minutes, and 193 mg (0.3 mmol) of bis(triphenylphosphine)palladium (II) dichloride was added thereto. The reaction mixture was stirred at 100° C. for 5 hours. After completion of the reaction, the reaction product was cooled to room temperature, diluted with ethyl acetate, and washed with distilled water and sodium bicarbonate aqueous solution. The organic layer thus separated was dried with anhydrous sodium sulfate and filtered under reduced pressure, and then the residues obtained by distillation under reduced pressure were separated by column chromatography (ethyl acetate:hexane at a volume ratio of 1:1), so as to obtain 525 mg (yield: 28%) of the title compound.

¹H-NMR (300 MHz, CDCl₃): δ 8.60˜8.54 (m, 2H), 8.20˜8.09 (m, 2H), 7.25˜7.17 (m, 2H), 7.05˜7.00 (m, 1H), 6.92˜6.88 (m, 1H), 5.70 (brs, 2H), 4.09 (s, 2H), 3.01˜2.92 (m, 1H), 2.74 (s, 3H), 2.15 (s, 3H), 1.41 (s, 9H), 1.28˜1.25 (d, 6H).

### Step 11) Preparation of N-(3-(6-amino-5-(3-(methylamino)prop-1-yn-1-yl)pyrimidin-4-yl)-5-fluoro-2-methylphenyl)-2-fluoro-4-isopropylbenzamide

515 mg (0.94 mmol) of the compound prepared in step 10) was diluted with 10 mL of dichloromethane, and 1.4 mL (18.7 mmol) of trifluoroacetic acid was slowly added thereto at 25° C. The reaction mixture was stirred at 25° C. for 1 hour. After completion of the reaction, the reaction product was distilled under reduced pressure, so as to obtain 421 mg (99%) of the title compound.

### Step 12) Preparation of N-(3-(6-amino-5-(3-N-methylacrylamido)prop-1-yn-1-yl)pyrimidin-4-yl)5-fluoro-2-methylphenyl)-2-fluoro-4-isopropylbenzamide

0.1 mL (1.4 mmol) of acrylic acid and 0.66 mL (3.7 mmol) of N,N-diisopropylethylamine were diluted with 4 mL of N,N-dimethylformamide, and 0.36 mL (1.2 mmol) of propylphosphonic anhydride (wt. 50%) was added thereto. The reaction mixture was stirred at 25° C. for 30 minutes.

421 mg (0.94 mmol) of the compound prepared in step 11) was diluted with 4 mL of N,N-dimethylformamide and cooled to 0° C. 0.5 mL (2.8 mmol) of N,N-diisopropylethylamine was added thereto, followed by stirring at the same temperature for 30 minutes. The resulting reaction mixture was slowly added dropwise and stirred at 0° C. for 4 hours. After completion of the reaction, the reaction product was diluted with ethyl acetate and washed with distilled water. The organic layer thus separated was dried with anhydrous sodium sulfate and filtered under reduced pressure, and then the residues obtained by distillation under reduced pressure were separated by column chromatography (dichloromethane:methanol at a volume ratio of 8:1), so as to obtain 100 mg (yield: 21%) of the title compound.

¹H-NMR (300 MHz, DMSO-d₆): δ 9.78 (bs, 1H), 8.40 (s, 1J), 7.71˜7.66 (t, 1H), 7.55˜7.51 (d, 1H), 7.26˜7.16 (m, 2H), 6.99˜6.97 (m, 1H), 6.68˜6.56 (m, 1H), 6.09˜5.99 (m, 1H), 5.65˜5.56 (m, 1H), 4.33 (s, 2H), 3.02˜2.93 (m, 1H), 2.80˜2.73 (m, 3H), 2.02 (s, 3H), 1.23˜1.21 (d, 6H).

MS (ESI⁺): m/z=504.21 [M+H]⁺.

### Example 2: Preparation of N-(3-(6-amino-5-(3-N-methylacrylamido)prop-1-yn-1-yl)pyrimidin-4-yl)5-fluoro-2-methylphenyl)-4-cyclopropyl-2-fluorobenzamide

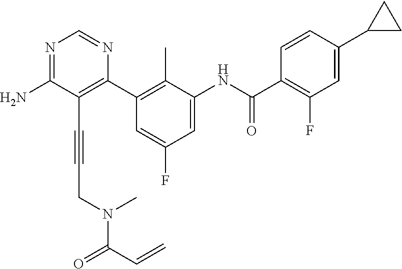 [SMILES: C=CC(=O)N(C)CC#Cc1c(N)ncnc1-c1cc(F)cc(NC(=O)c2ccc(C3CC3)cc2F)c1C]

213 mg (yield: 38%) of the title compound was prepared in the same manner as in Example 1, except that 4-cyclopropyl-2-fluoro-N-(5-fluoro-2-methyl-3-(4,4,5,5,-tetramethyl-1,3,2-dioxaborolan-2-yl)phenyl)benzamide was used instead of 2-fluoro-N-(5-fluoro-2-methyl-3-(4,4,5,5-tetramethyl-1,3,2-dioxaborolan-2-yl)phenyl)-4-isopropylbenzamide in step 10) of Example 1.

¹H-NMR (300 MHz, DMSO-d₆) δ 9.71 (bs, 1H), 8.39 (s, 1H), 7.66˜7.61 (t, 1H), 7.53˜7.50 (m, 1H), 7.07˜7.02 (m, 2H), 6.98˜6.96 (m, 1H), 6.66˜6.55 (m, 1H), 6.09˜5.98 (m, 1H), 5.64˜5.55 (m, 1H), 4.33 (s, 2H), 2.79˜2.72 (m, 3H), 2.05˜1.96 (m, 4H), 1.06˜1.00 (m, 1H), 0.80˜0.76 (m, 1H).

MS (ESI⁺): m/z=502.20 [M+H]⁺.

### Example 3: Preparation of N-(3-(4-amino-6-(3-(6-cyclopropyl-8-fluoro-1-oxo-3,4-dihydroisoquinolin-2(1H)-yl)-5-fluorophenyl)pyrimidin-5-yl)prop-2-yn-1-yl)-N-methylacrylamide

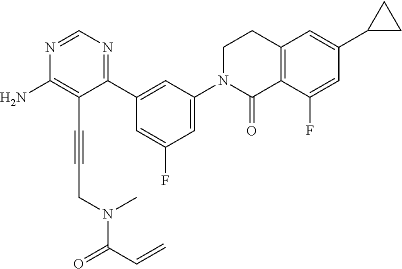 [SMILES: C=CC(=O)N(C)CC#Cc1c(N)ncnc1-c1cc(F)cc(N2CCc3cc(C4CC4)cc(F)c3C2=O)c1]

64 mg (yield: 12%) of the title compound was prepared in the same manner as in Example 1, except that 6-cyclopropyl-8-fluoro-2-(3-fluoro-5-(4,4,5,5-tetramethyl-1,3,2-dioxaborolan-2-yl)phenyl-3,4-dihydroisoquinolin-1(2H)-one was used instead of 2-fluoro-N-(5-fluoro-2-methyl-3-(4,4,5,5-tetramethyl-1,3,2-dioxaborolan-2-yl)phenyl)-4-isopropylbenzamide in step 10) of Example 1.

¹H-NMR (300 MHz, DMSO-d₆) δ 8.42 (s, 1H), 7.83 (m, 1H), 7.61 (m, 1H), 7.43 (d, 1H), 6.92 (s, 1H), 6.90 (d, 1H), 6.83 (m, 1H), 6.11 (d, 1H), 5.62 (m, 1H), 4.55 (d, 2H), 3.93 (m, 2H), 3.09 (m, 1H), 3.07 (s, 3H), 2.88 (s, 1H), 2.00 (m, 1H), 1.05 (m, 2H), 0.82 (m, 2H).

MS (ESI⁺): m/z=514.20 [M+H]⁺.

### Example 4: Preparation of N-(3-(4-amino-6-(3-(6-cyclopropyl-8-fluoro-1-oxo-3,4-dihydroisoquinolin-2(1H)-yl)-5-fluoro-2-methylphenyl)pyrimidin-5-yl)prop-2-yn-1-yl)-N-methylacrylamide

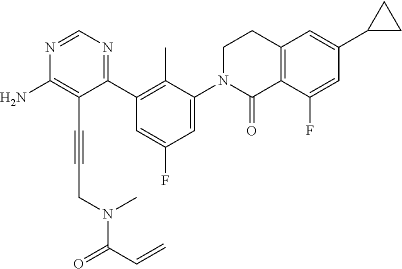 [SMILES: C=CC(=O)N(C)CC#Cc1c(N)ncnc1-c1cc(F)cc(N2CCc3cc(C4CC4)cc(F)c3C2=O)c1C]

10 mg (yield: 11%) of the title compound was prepared in the same manner as in Example 1, except that 6-cyclopropyl-8-fluoro-2-(5-fluoro-2-methyl-3-(4,4,5,5-tetramethyl-1,3,2-dioxaborolan-2-yl)phenyl)-3,4-dihydroisoquinoline-1(2H)-one was used instead of 2-fluoro-N-(5-fluoro-2-methyl-3-(4,4,5,5-tetramethyl-1,3,2-dioxaborolan-2-yl)phenyl)-4-isopropylbenzamide in step 10) of Example 1.

¹H-NMR (300 MHz, DMSO-d₆) δ 8.41 (s, 1H), 7.34 (m, 1H), 7.13 (m, 1H), 6.94 (s, 1H), 6.86 (d, 1H), 6.74 (m, 1H), 6.13 (d, 1H), 5.61 (m, 1H), 4.36 (d, 2H), 3.87 (m, 1H), 3.61 (m, 1H), 3.08 (s, 3H), 2.83 (s, 1H), 2.77 (s, 1H), 2.01 (m, 1H), 1.95 (s, 3H), 1.05 (m, 2H), 0.83 (m, 2H).

MS (ESI⁺): m/z=528.21 [M+H]⁺.

### Example 5: Preparation of N-(3-(4-amino-6-(3-(6-cyclo-8-fluoro-1-oxoisoquinolin-2(1H)-yl)-5-fluorophenyl)pyrimidin-5-yl)prop-2-yn-1-yl)-N-methylacrylamide

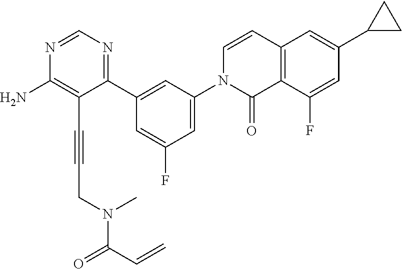 [SMILES: C=CC(=O)N(C)CC#Cc1c(N)ncnc1-c1cc(F)cc(-n2ccc3cc(C4CC4)cc(F)c3c2=O)c1]

103 mg (yield: 28%) of the title compound was prepared in the same manner as in Example 1, except that 6-cyclopropyl-8-fluoro-2-(3-fluoro-5-(4,4,5,5-tetramethyl-1,3,2-dioxaborolan-2-yl)phenyl)isoquinolin-1(2H)-one was used instead of 2-fluoro-N-(5-fluoro-2-methyl-3-(4,4,5,5-tetramethyl-1,3,2-dioxaborolan-2-yl)phenyl)-4-isopropylbenzamide in step 10) of Example 1.

¹H-NMR (300 MHz, DMSO-d₆) δ 8.43 (s, 1H), 7.89˜7.84 (m, 2H), 7.59˜7.57 (m, 1H), 7.56˜7.48 (m, 1H), 7.02˜6.97 (d, 1H), 6.65˜6.61 (d, 1H), 6.11˜6.05 (dd, 1H), 5.63˜5.59 (dd, 1H), 4.55˜4.48 (d, 2H), 3.01 (s, 2H), 2.85 (s, 1H), 2.10˜2.04 (m, 1H), 1.13˜1.06 (m, 2H), 0.90˜0.86 (m, 2H).

MS (ESI⁺): m/z=512.18 [M+H]⁺.

### Example 6: Preparation of N-(3-(4-amino-6-(3-(6-cyclopropyl-8-fluoro-1-oxoisoquinolin-2(1H)-yl)-5-fluoro-2-methylphenyl)pyrimidin-5-yl)prop-2-yn-1-yl)-N-methylacrylamide

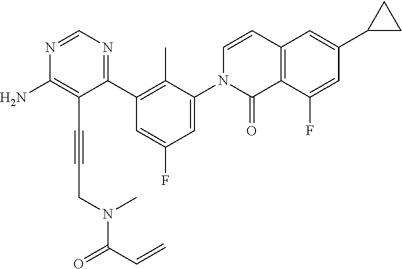 [SMILES: C=CC(=O)N(C)CC#Cc1c(N)ncnc1-c1cc(F)cc(-n2ccc3cc(C4CC4)cc(F)c3c2=O)c1C]

87 mg (yield: 17%) of the title compound was prepared in the same manner as in Example 1, except that 6-cyclopropyl-8-fluoro-2-(5-fluoro-2-methyl-3-(4,4,5,5-tetramethyl-1,3,2-dioxaborolan-2-yl)phenyl)isoquinolin-1(2H)-one was used instead of 2-fluoro-N-(5-fluoro-2-methyl-3-(4,4,5,5-tetramethyl-1,3,2-dioxaborolan-2-yl)phenyl)-4-isopropylbenzamide in step 10) of Example 1.

¹H-NMR (300 MHz, DMSO-d₆) δ 8.40 (s, 1H), 7.38˜7.35 (m, 1H), 7.27˜7.21 (m, 3H), 7.01˜6.96 (m, 1H), 6.74˜6.60 (m, 2H), 6.13˜6.07 (m, 1H), 5.66˜5.56 (m, 1H), 4.39˜4.35 (m, 2H), 2.89˜2.79 (m, 3H), 2.09˜2.01 (m, 1H), 1.80 (s, 3H), 1.11˜1.04 (m, 2H), 0.87˜0.85 (m, 2H).

MS (ESI⁺): m/z=526.20 [M+H]⁺.

### Example 7: Preparation of N-(3-(4-amino-6-(3-(6-cyclopropyl-8-fluoro-1-oxoisoquinolin-2(1H)-yl)-2,5-difluorophenyl)pyrimidin-5-yl)prop-2-yn-1-yl)-N-methylacrylamide

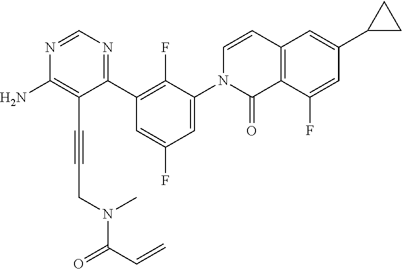 [SMILES: C=CC(=O)N(C)CC#Cc1c(N)ncnc1-c1cc(F)cc(-n2ccc3cc(C4CC4)cc(F)c3c2=O)c1F]

3 mg (yield: 5%) of the title compound was prepared in the same manner as in Example 1, except that 6-cyclopropyl-2-(2,5-difluoro-3-(4,4,5,5-tetramethyl-1,3,2-dioxaborolan-2-yl)phenyl)-8-fluoroisoquinolin-1(2H)-one was used instead of 2-fluoro-N-(5-fluoro-2-methyl-3-(4,4,5,5-tetramethyl-1,3,2-dioxaborolan-2-yl)phenyl)-4-isopropylbenzamide in step 10) of Example 1.

¹H-NMR (300 MHz, DMSO-d₆) δ 8.43 (s, 1H), 7.69 (m, 1H), 7.51 (m, 1H), 7.42 (m, 1H), 7.26 (s, 1H), 7.02 (m, 1H), 6.71 (m, 1H), 6.06 (d, 1H), 5.64 (m, 1H), 4.46 (d, 2H), 3.35 (m, 2H), 3.30 (s, 3H), 2.92 (s, 1H), 2.80 (s, 1H), 2.06 (m, 1H), 1.10 (m, 2H), 0.85 (m, 2H).

MS (ESI⁺): m/z=530.17 [M+H]⁺.

### Example 8: Preparation of N-(3-(4-amino-6-(3-(6-cyclopropyl-8-fluoro-1-oxoisoquinolin-2(1H)-yl)-2,5-dimethylphenyl)pyrimidin-5-yl)prop-2-yn-1-yl)-N-methylacrylamide

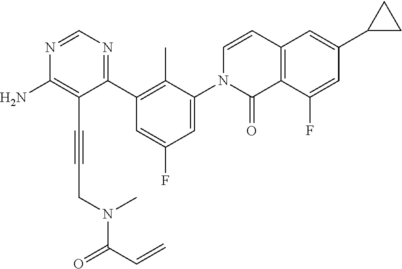 [SMILES: C=CC(=O)N(C)CC#Cc1c(N)ncnc1-c1cc(F)cc(-n2ccc3cc(C4CC4)cc(F)c3c2=O)c1C]

18 mg (yield: 11%) of the title compound was prepared in the same manner as in Example 1, except that 6-cyclopropyl-2-(2,5-dimethyl-3-(4,4,5,5-tetramethyl-1,3,2-dioxaborolan-2-yl)phenyl)-8-fluoroisoquinolin-1(2H)-one was used instead of 2-fluoro-N-(5-fluoro-2-methyl-3-(4,4,5,5-tetramethyl-1,3,2-dioxaborolan-2-yl)phenyl)-4-isopropylbenzamide in step 10) of Example 1.

¹H-NMR (300 MHz, DMSO-d₆) δ 8.40 (s, 1H), 7.26˜7.16 (m, 4H), 7.00˜6.96 (d, 1H), 6.76˜6.60 (m, 2H), 6.28˜5.91 (m, 1H), 5.72˜5.57 (dd, 1H), 4.40˜4.21 (d, 2H), 2.88˜2.78 (d, 3H), 2.32 (s, 3H), 2.09˜2.04 (m, 1H), 1.81 (s, 3H), 1.12˜1.06 (m, 2H), 0.89˜0.84 (m, 2H).

MS (ESI⁺): m/z=522.22 [M+H]⁺.

### Example 9: Preparation of N-(3-(4-amino-6-(3-(6-cyclopropyl-8-fluoro-1-oxoisoquinolin-2(1H)-yl)-5-fluoro-2-methylphenyl)pyrimidin-5-yl)prop-2-yn-1-yl)acrylamide

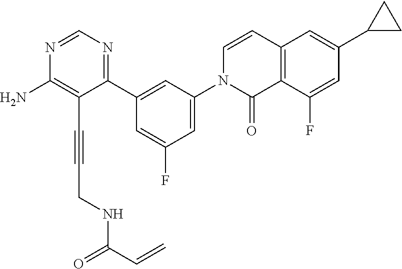 [SMILES: C=CC(=O)NCC#Cc1c(N)ncnc1-c1cc(F)cc(-n2ccc3cc(C4CC4)cc(F)c3c2=O)c1]

30 mg (yield: 6%) of the title compound was prepared in the same manner as in Example 1, except that tert-butyl (3-(4-amino-6-chloropyrimidin-5-yl)prop-2-yn-1-yl)carbamate was used instead of tert-butyl (3-(4-amino-6-chloropyrimidin-5-yl)prop-2-yn-1-yl)(methyl)carbamate and 6-cyclopropyl-8-fluoro-2-(5-fluoro-2-methyl-3-(4,4,5,5-tetramethyl-1,3,2-dioxaborolan-2-yl)phenyl)isoquinolin-1(2H)-one was used instead of 2-fluoro-N-(5-fluoro-2-methyl-3-(4,4,5,5-tetramethyl-1,3,2-dioxaborolan-2-yl)phenyl)-4-isopropylbenzamide in step 10) of Example 1.

¹H-NMR (300 MHz, DMSO-d₆) δ 8.43˜8.41 (m, 2H), 7.40˜7.32 (m, 2H), 7.27˜7.22 (m, 2H), 7.02˜6.97 (m, 1H), 6.65˜6.62 (m, 1H), 6.25˜6.06 (m, 2H), 5.63 (5.59 (m, 1H), 4.20˜4.10 (m, 2H), 2.09˜2.03 (m, 1H), 1.82 (s, 3H), 1.12˜1.05 (m, 2H), 0.88˜0.83 (m, 2H).

MS (ESI⁺): m/z=512.18 [M+H]⁺.

### Example 10: Preparation of N-(3-(4-amino-6-(3-(6-(tert-butyl)-8-fluoro-1-oxophthalazin-2-(1H)-yl)-5-fluorophenyl)pyrimidin-5-yl)prop-2-yn-1-yl)-N-methylacrylamide

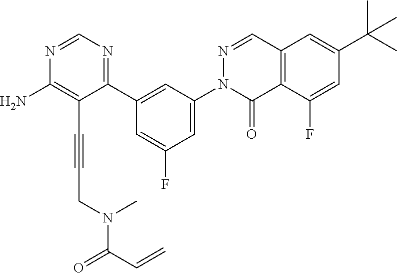 [SMILES: C=CC(=O)N(C)CC#Cc1c(N)ncnc1-c1cc(F)cc(-n2ncc3cc(C(C)(C)C)cc(F)c3c2=O)c1]

89 mg (yield: 23%) of the title compound was prepared in the same manner as in Example 1, except that 6-(tert-butyl)-8-fluoro-2-(3-fluoro-5-(4,4,5,5-tetramethyl-1,3,2-dioxaborolan-2-yl)phenyl)phthalazin-1(2H)-one was used instead of 2-fluoro-N-(5-fluoro-2-methyl-3-(4,4,5,5-tetramethyl-1,3,2-dioxaborolan-2-yl)phenyl)-4-isopropylbenzamide in step 10) of Example 1.

¹H-NMR (300 MHz, DMSO-d₆) δ 8.56 (s, 1H), 8.44 (s, 1H), 8.14˜8.11 (m, 1H), 7.87˜7.86 (d, 1H), 7.83˜7.74 (m, 2H), 7.68˜7.63 (td, 1H), 6.84˜6.57 (m, 1H), 6.06˜6.00 (td, 1H), 5.61˜5.57 (d, 1H), 4.55˜4.48 (d, 2H), 3.05˜2.85 (d, 3H), 1.38 (s, 9H).

MS (ESI⁺): m/z=515.19 [M+H]⁺.

### Example 11: Preparation of N-(3-(4-amino-6-(3-(6-cyclopropyl-1-oxophthalazin-2(1H)-yl)-5-fluoro-2-methylphenyl)pyrimidin-5-yl)prop-2-yn-1-yl)-N-methylacrylamide

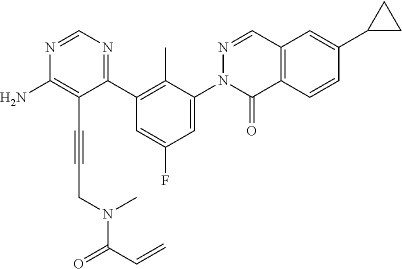 [SMILES: C=CC(=O)N(C)CC#Cc1c(N)ncnc1-c1cc(F)cc(-n2ncc3cc(C4CC4)ccc3c2=O)c1C]

63 mg (yield: 15%) of the title compound was prepared in the same manner as in Example 1, except that 6-cyclopropyl-2-(5-fluoro-2-methyl-3-(4,4,5,5-tetramethyl-1,3,2-dioxaborolan-2-yl)phenyl)phthalazin-1(2H)-one was used instead of 2-fluoro-N-(5-fluoro-2-methyl-3-(4,4,5,5-tetramethyl-1,3,2-dioxaborolan-2-yl)phenyl)-4-isopropylbenzamide in step 10) of Example 1.

¹H-NMR (300 MHz, DMSO-d₆) δ 8.45 (s, 1H), 8.40 (s, 1H), 8.18˜8.15 (m, 1H), 7.69˜7.61 (m, 1H), 7.41˜7.38 (m, 1H), 7.28˜7.23 (m, 1H), 6.73˜6.64 (m, 1H), 6.12˜6.01 (m, 1H), 5.69˜5.58 (m, 1H), 4.38 (s, 2H), 2.87˜2.79 (m, 3H), 2.22˜2.13 (m, 1H), 1.81 (s, 3H), 1.17˜1.10 (m, 2H), 0.90˜0.87 (m, 2H).

MS (ESI⁺): m/z=509.20 [M+H]⁺.

### Example 12: Preparation of N-(3-(4-amino-6-(3-(6-cyclopropyl-8-fluoro-1-oxophthalazin-2(1H)-yl)-5-fluoro-2-methylphenyl)pyrimidin-6-yl)prop-2-yn-1-yl)-N-methylacrylamide

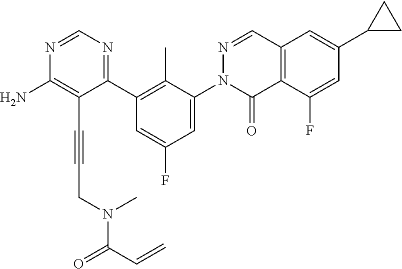 [SMILES: C=CC(=O)N(C)CC#Cc1c(N)ncnc1-c1cc(F)cc(-n2ncc3cc(C4CC4)cc(F)c3c2=O)c1C]

32 mg (yield: 22%) of the title compound was prepared in the same manner as in Example 1, except that 6-cyclopropyl-8-fluoro-2-(5-fluoro-2-methyl-3-(4,4,5,5-tetramethyl-1,3,2-dioxaborolan-2-yl)phenyl)phthalazin-1(2H)-one was used instead of 2-fluoro-N-(5-fluoro-2-methyl-3-(4,4,5,5-tetramethyl-1,3,2-dioxaborolan-2-yl)phenyl)-4-isopropylbenzamide in step 10) of Example 1.

¹H-NMR (300 MHz, DMSO-d₆) δ 8.42˜8.41 (m, 2H), 7.55 (s, 1H), 7.44˜7.38 (m, 2H), 7.28˜7.23 (m, 1H), 6.73˜6.62 (m, 1H), 6.11˜6.01 (m, 1H), 5.68˜5.58 (m, 1H), 4.38 (s, 2H), 2.87˜2.84 (m, 3H), 2.20˜2.11 (m, 1H), 1.82 (s, 3H), 1.18˜1.11 (m, 2H), 0.94˜0.88 (m, 2H).

MS (ESI⁺): m/z=527.19 [M+H]⁺.

### Example 13: Preparation of N-(3-(4-amino-(3-(6-(tert-butyl)-8-fluoro-1-oxophthalazin-2(1H)-yl)-5-fluoro-2-methylphenyl)pyrimidin-6-yl)prop-2-yn-1-yl)-N-methylacrylamide

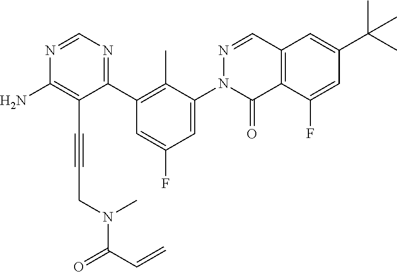 [SMILES: C=CC(=O)N(C)CC#Cc1c(N)ncnc1-c1cc(F)cc(-n2ncc3cc(C(C)(C)C)cc(F)c3c2=O)c1C]

18 mg (yield: 22%) of the title compound was prepared in the same manner as in Example 1, except that 6-(tert-butyl)-8-fluoro-2-(5-fluoro-2-methyl-3-(4,4,5,5-tetramethyl-1,3,2-dioxaborolan-2-yl)phenyl)phthalazin-1(2H)-one was used instead of 2-fluoro-N-(5-fluoro-2-methyl-3-(4,4,5,5-tetramethyl-1,3,2-dioxaborolan-2-yl)phenyl)-4-isopropylbenzamide in step 10) of Example 1.

¹H-NMR (300 MHz, DMSO-d₆) δ 8.54˜8.53 (m, 1H), 8.42 (s, 1H), 7.88 (s, 1H), 7.78˜7.73 (m, 1H), 7.42˜7.39 (m, 1H), 7.29˜7.25 (m, 1), 6.75˜6.64 (m, 1H), 6.12˜6.03 (m, 1H), 5.70˜5.60 (m, 1H), 4.39 (s, 2H), 2.87˜2.81 (m, 3H), 1.85 (s, 3H), 1.37 (s, 9H).

MS (ESI⁺): m/z=543.22 [M+H]⁺.

### Example 14: Preparation of N-(3-(4-amino-6-(3-(6-tert-butyl)-1-oxophthalazin-2-(1H)-yl)-5-fluorophenyl)pyrimidin-5-yl)prop-2-yn-1-yl)-N-methylacrylamide

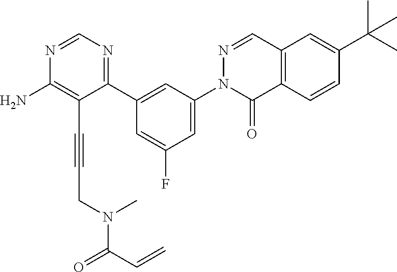 [SMILES: C=CC(=O)N(C)CC#Cc1c(N)ncnc1-c1cc(F)cc(-n2ncc3cc(C(C)(C)C)ccc3c2=O)c1]

50 mg (yield: 20%) of the title compound was prepared in the same manner as in Example 1, except that 6-(tert-butyl)-2-(3-fluoro-5-(4,4,5,5-tetramethyl-1,3,2-dioxaborolan-2-yl)phenyl)phthalazin-1(2H)-one was used instead of 2-fluoro-N-(5-fluoro-2-methyl-3-(4,4,5,5-tetramethyl-1,3,2-dioxaborolan-2-yl)phenyl)-4-isopropylbenzamide in step 10) of Example 1.

¹H-NMR (300 MHz, DMSO-d₆) δ 8.51 (s, 1H), 8.44 (s, 1H), 8.22-8.19 (d, 1H), 8.16 (s, 1H), 7.82-7.76 (t, 1H), 7.70-7.63 (m, 3H), 6.74-6.54 (m, 1H), 6.07-6.01 (dd, 1H), 5.61-5.56 (dd, 1H), 4.54-4.49 (d, 2H), 3.00-2.83 (d, 3H), 2.27-2.17 (m, 1H), 1.19-1.13 (m, 2H), 0.93-0.89 (m, 2H).

MS (ESI⁺): m/z=511.22 [M+H]⁺.

### Example 15: Preparation of N-(3-(4-amino-6-(3-(7-cyclopropyl-5-fluoro-4-oxoquinazolin-3(4H)-yl)-5-fluorophenyl)pyrimidin-5-yl)prop-2-yn-1-yl)-N-methylacrylamide

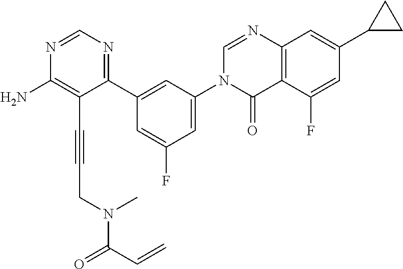 [SMILES: C=CC(=O)N(C)CC#Cc1c(N)ncnc1-c1cc(F)cc(-n2cnc3cc(C4CC4)cc(F)c3c2=O)c1]

26 mg (yield: 14%) of the title compound was prepared in the same manner as in Example 1, except that 7-cyclopropyl-5-fluoro-3-(3-fluoro-5-(4,4,5,5-tetramethyl-1,3,2-dioxaborolan-2-yl)phenyl)quinazolin-4(3H)-one was used instead of 2-fluoro-N-(5-fluoro-2-methyl-3-(4,4,5,5-tetramethyl-1,3,2-dioxaborolan-2-yl)phenyl)-4-isopropylbenzamide in step 10) of Example 1.

¹H-NMR (300 MHz, DMSO-d₆) δ 8.53 (s, 1H), 8.48 (s, 1H), 8.42-8.39 (m, 1H), 8.17 (s, 1H), 7.80-7.73 (m, 1H), 7.71-7.66 (m, 3H), 6.70-5.83 (m, 2H), 5.65-5.51 (d, 1H), 4.52-4.46 (d, 2H), 3.01-2.83 (d, 3H), 2.26-2.15 (m, 1H), 1.18-1.13 (m, 2H), 0.93-0.90 (m, 2H).

MS (ESI⁺): m/z=513.18 [M+H]⁺.

### Example 16: Preparation of (R)-2-(3-(5-((1-acryloylpyrrolidin-2-yl)ethynyl)-6-aminopyrimidin-4-yl)-5-fluoro-2-methylphenyl)-6-cyclopropyl-8-fluoroisoquinolin-1-(2H)-one

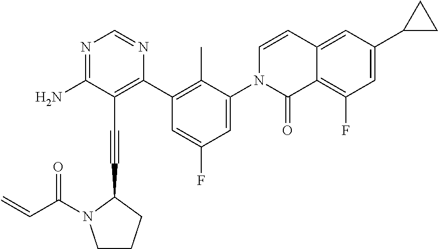 [SMILES: C=CC(=O)N1CCC[C@@H]1C#Cc1c(N)ncnc1-c1cc(F)cc(-n2ccc3cc(C4CC4)cc(F)c3c2=O)c1C]

40 mg (yield: 21%) of the title compound was prepared in the same manner as in Example 1, except that tert-butyl (R)-2-((4-amino-6-chloropyrimidin-5-yl)ethynyl)pyrrolidine-1-carboxylate was used instead of tert-butyl (3-(4-amino-6-chloropyrimidin-5-yl)prop-2-yn-1-yl)(methyl)carbamate and 6-cyclopropyl-8-fluoro-2-(5-fluoro-2-methyl-3-(4,4,5,5-tetramethyl-1,3,2-dioxaborolan-2-yl)phenyl)isoquinolin-1(2H)-one was used instead of 2-fluoro-N-(5-fluoro-2-methyl-3-(4,4,5,5-tetramethyl-1,3,2-dioxaborolan-2-yl)phenyl)-4-isopropylbenzamide in step 10) of Example 1.

¹H-NMR (300 MHz, DMSO-d₆) δ 8.42 (s, 1H), 7.41˜7.24 (m, 4H), 7.02˜6.98 (d, 1H), 6.66˜6.64 (m, 1H), 6.54˜6.51 (m, 1H), 6.18˜6.07 (m, 1H), 5.68˜5.48 (m, 1H), 4.96˜4.95 (m, 1H), 4.77˜4.75 (m, 1H), 3.45˜3.32 (m, 2H), 2.13˜2.03 (m, 3H), 1.90˜1.89 (m, 2H), 1.13˜1.06 (m, 2H), 0.89˜0.86 (m, 2H).

MS (ESI⁺): m/z=552.21 [M+H]⁺.

### Example 17: Preparation of (S)-2-(3-(5-((1-acryloylpyrrolidin-2-yl)ethynyl)-6-aminopyrimidin-4-yl)-6-fluoro-2-methylphenyl)-6-cyclopropyl-8-fluoroisoquinolin-1-(2H)-one

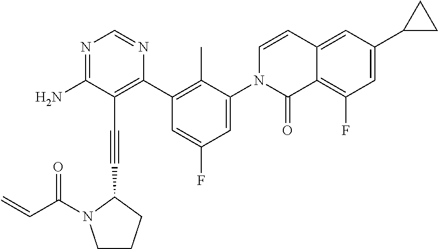 [SMILES: C=CC(=O)N1CCC[C@H]1C#Cc1c(N)ncnc1-c1cc(F)cc(-n2ccc3cc(C4CC4)cc(F)c3c2=O)c1C]

42 mg (yield: 25%) of the title compound was prepared in the same manner as in Example 1, except that tert-butyl (S)-2-((4-amino-6-chloropyrimidin-5-yl)ethynyl)pyrrolidine-1-carboxylate was used instead of tert-butyl (3-(4-amino-6-chloropyrimidin-5-yl)prop-2-yn-1-yl)(methyl)carbamate and 6-cyclopropyl-8-fluoro-2-(5-fluoro-2-methyl-3-(4,4,5,5-tetramethyl-1,3,2-dioxaborolan-2-yl)phenyl)isoquinolin-1(2H)-one was used instead of 2-fluoro-N-(5-fluoro-2-methyl-3-(4,4,5,5-tetramethyl-1,3,2-dioxaborolan-2-yl)phenyl)-4-isopropylbenzamide in step 10) of Example 1.

¹H-NMR (300 MHz, DMSO-d₆) δ 8.42 (s, 1H), 7.41˜7.24 (m, 4H), 7.02˜6.98 (d, 1H), 6.66˜6.64 (m, 1H), 6.54˜6.51 (m, 1H), 6.18˜6.07 (m, 1H), 5.68˜5.48 (m, 1H), 4.96˜4.95 (m, 1H), 4.77˜4.75 (m, 1H), 3.45˜3.32 (m, 2H), 2.13˜2.03 (m, 3H), 1.90˜1.89 (m, 2H), 1.13˜1.06 (m, 2H), 0.89˜0.86 (m, 2H).

MS (ESI⁺): m/z=552.21 [M+H]⁺.

### Example 18: Preparation of (S)-2-(3-(5-((1-acryloylpyrrolidin-2-yl)ethynyl)-6-aminopyrimidin-4-yl)-6-fluoro-2-methylphenyl)-8-fluoro-6-isoquinolin-1-(2H)-one

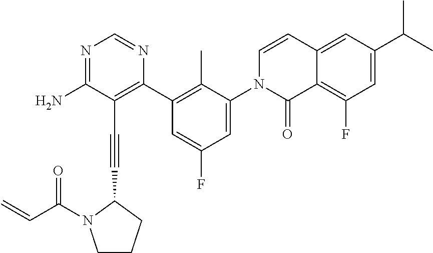 [SMILES: C=CC(=O)N1CCC[C@H]1C#Cc1c(N)ncnc1-c1cc(F)cc(-n2ccc3cc(C(C)C)cc(F)c3c2=O)c1C]

16 mg (yield: 22%) of the title compound was prepared in the same manner as in Example 1, except that 8-fluoro-2-(5-fluoro-2-methyl-3-(4,4,5,5-tetramethyl-1,3,2-dioxaborolan-2-yl)phenyl)isopropylisoquinolin-1(2H)-one was used instead of 6-cyclopropyl-8-fluoro-2-(5-fluoro-2-methyl-3-(4,4,5,5-tetramethyl-1,3,2-dioxaborolan-2-yl)phenyl)-4-isopropylbenzamide in step 10) of Example 1.

¹H-NMR (300 MHz, DMSO-d₆) δ 8.43 (s, 1H), 7.44˜7.20 (m, 5H), 6.73˜6.53 (m, 2H), 6.20˜6.02 (m, 1H), 5.76˜5.69 (m, 1H), 4.96˜4.76 (m, 1H), 3.15˜3.01 (m, 1H), 2.25˜1.75 (m, 6H), 1.35˜1.20 (d, 6H).

MS (ESI⁺): m/z=554.23 [M+H]⁺.

### Example 19: Preparation of (S)-2-(3-(6-amino-5-((1-(2-fluoroacryloyl)pyrrolidin-2-yl)ethynyl)pyrimidin-4-yl)-5-fluoro-2-methylphenyl)-6-cyclopropyl-8-fluoroisoquinolin-1(2H)-one

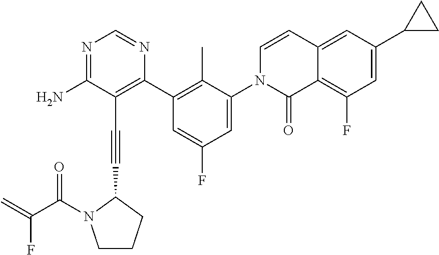 [SMILES: C=C(F)C(=O)N1CCC[C@H]1C#Cc1c(N)ncnc1-c1cc(F)cc(-n2ccc3cc(C4CC4)cc(F)c3c2=O)c1C]

19 mg (yield: 5%) of the title compound was prepared in the same manner as in Example 12, except that 2-fluoroacrylic acid was used instead of acrylic acid in Example 12.

¹H-NMR (300 MHz, DMSO-d₆) δ 8.41 (s, 1H), 7.40˜7.23 (m, 4H), 7.00˜6.96 (m, 1H), 6.64˜6.60 (m, 1H), 5.46˜5.28 (m, 2H), 4.77 (m, 1H), 4.43˜4.33 (m, 1H), 3.57˜3.41 (m, 2H), 2.09˜2.01 (m, 2H), 1.85˜1.82 (m, 6H), 1.11˜1.04 (m, 2H), 0.87˜0.82 (m, 2H).

MS (ESI⁺): m/z=570.20 [M+H]⁺.

### Example 20: Preparation of (S)-2-(3-(6-amino-5-((1-(2-chloroacryloyl)pyrrolidin-2-yl)ethynyl)pyrimidin-4-yl)-5-fluoro-2-methylphenyl)-6-cyclopropyl-8-fluoroisoquinolin-1(2H)-one

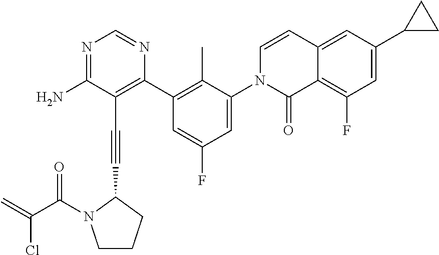 [SMILES: C=C(Cl)C(=O)N1CCC[C@H]1C#Cc1c(N)ncnc1-c1cc(F)cc(-n2ccc3cc(C4CC4)cc(F)c3c2=O)c1C]

45 mg (yield: 11%) of the title compound was prepared in the same manner as in Example 12, except that 2-chloroacrylic acid was used instead of acrylic acid in Example 12.

¹H-NMR (300 MHz, DMSO-d₆) δ 8.42 (s, 1H), 7.42˜7.26 (m, 4H), 7.01˜6.96 (m, 1H), 6.64˜6.60 (m, 1H), 5.83˜5.74 (m, 2H), 4.75 (m, 1H), 4.43˜4.34 (m, 1H), 3.51˜3.42 (m, 2H), 2.13˜2.01 (m, 2H), 1.97˜1.82 (m, 6H), 1.11˜1.05 (m, 2H), 0.88˜0.83 (m, 2H).

MS (ESI⁺): m/z=586.17 [M+H]⁺.

### Example 21: Preparation of (S)—N-(3-(5-((1-acryloylpyrrolidin-2-yl)ethynyl)-6-aminopyrimidin-4-yl)-6-fluoro-2-methylphenyl)-4-cyclopropyl-2-fluorobenzamide

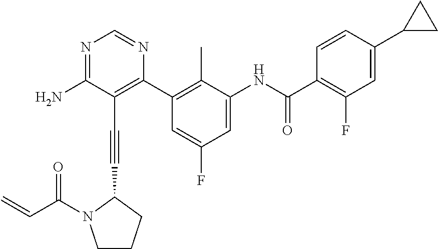 [SMILES: C=CC(=O)N1CCC[C@H]1C#Cc1c(N)ncnc1-c1cc(F)cc(NC(=O)c2ccc(C3CC3)cc2F)c1C]

225 mg (yield: 31%) of the title compound was prepared in the same manner as in Example 17, except that 4-cyclopropyl-2-fluoro-N-(5-fluoro-2-methyl-3-(4,4,5,5,-tetramethyl-1,3,2-dioxaborolan-2-yl)phenyl)benzamide was used instead of 6-cyclopropyl-8-fluoro-2-(5-fluoro-2-methyl-3-(4,4,5,5-tetramethyl-1,3,2-dioxaborolan-2-yl)phenyl)isoquinolin-1(2H)-one in step 10) of Example 17.

¹H-NMR (300 MHz, DMSO-d₆) δ 9.70-9.68 (m, 1H), 8.39-8.38 (d, 2H), 7.65-7.64 (m, 1H), 7.61-7.51 (m, 1H), 7.08-7.03 (m, 2H), 6.95-6.94 (m, 1H), 6.52-6.43 (m, 1H), 6.13-6.04 (m, 1H), 5.64-5.53 (m, 1H), 4.87-4.68 (m, 1H), 3.79-3.76 (m, 1H), 2.04-1.95 (m, 3H), 1.79-1.72 (m, 4H), 1.07-1.00 (m, 2H), 0.80-0.75 (m, 2H).

MS (ESI⁺): m/z=527.22 [M+H]⁺.

### Example 22: Preparation of 2-(3-(5-((1-acryloylpiperidin-2-yl)ethynyl)-6-aminopyrimidin-4-yl)-5-fluoro-2-methylphenyl)-6-cyclopropyl-8-isoquinolin-1(2H)-one

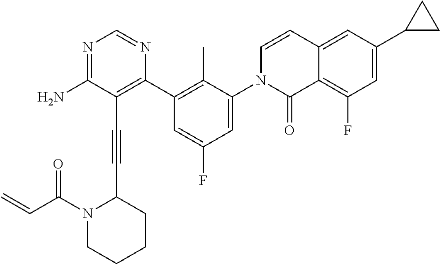 [SMILES: C=CC(=O)N1CCCCC1C#Cc1c(N)ncnc1-c1cc(F)cc(-n2ccc3cc(C4CC4)cc(F)c3c2=O)c1C]

25 mg (yield: 13%) of the title compound was prepared in the same manner as in Example 1, except that tert-butyl 2-((4-amino-6-chloropyrimidin-5-yl)ethynyl)piperidin-1-carboxylate was used instead of tert-butyl (3-(4-amino-6-chloropyrimidin-5-yl)prop-2-yn-1-yl)(methyl)carbamate and 6-cyclopropyl-8-fluoro-2-(5-fluoro-2-methyl-3-(4,4,5,5-tetramethyl-1,3,2-dioxaborolan-2-yl)phenyl)isoquinolin-1(2H)-one was used instead of 2-fluoro-N-(5-fluoro-2-methyl-3-(4,4,5,5-tetramethyl-1,3,2-dioxaborolan-2-yl)phenyl)-4-isopropylbenzamide in step 10) of Example 1.

¹H-NMR (300 MHz, DMSO-d₆) δ 8.42 (s, 1H), 7.43˜7.39 (m, 2H), 7.35 (s, 1H), 7.34˜7.19 (m, 1H), 7.02˜6.98 (d, 1H), 6.80˜6.71 (q, 1H), 6.65˜6.63 (m, 1H), 6.11˜6.04 (d, 1H), 5.70˜5.62 (t, 1H), 3.80˜3.73 (m, 1H), 2.85˜2.70 (m, 1H), 2.10˜2.03 (m, 1H), 1.80˜1.74 (m, 3H), 1.58˜1.52 (m, 4H) 1.11˜1.06 (t, 2H), 0.88˜0.85 (m, 2H).

MS (ESI⁺): m/z=566.23 [M+H]⁺.

### Example 23: Preparation of (S)-2-(3-(5-((1-acryloylpiperidin-2-yl)ethynyl)-6-aminopyrimidin-4-yl)-5-fluoro-2-methylphenyl)-8-fluoro-6-isopropylisoquinolin-1(2H)-one

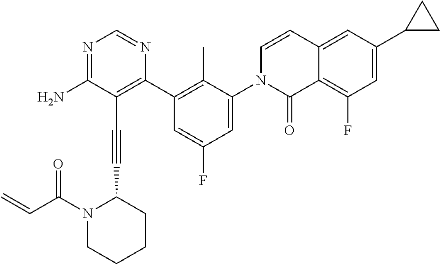 [SMILES: C=CC(=O)N1CCCC[C@H]1C#Cc1c(N)ncnc1-c1cc(F)cc(-n2ccc3cc(C4CC4)cc(F)c3c2=O)c1C]

55 mg (yield: 16%) of the title compound was prepared in the same manner as in Example 1, except that tert-butyl (S)-2-((4-amino-6-chloropyrimidin-5-yl)ethynyl)piperidin-1-carboxylate was used instead of tert-butyl (3-(4-amino-6-chloropyrimidin-5-yl)prop-2-yn-1-yl)(methyl)carbamate and 6-cyclopropyl-8-fluoro-2-(5-fluoro-2-methyl-3-(4,4,5,5-tetramethyl-1,3,2-dioxaborolan-2-yl)phenyl)isoquinolin-1(2H)-one was used instead of 2-fluoro-N-(5-fluoro-2-methyl-3-(4,4,5,5-tetramethyl-1,3,2-dioxaborolan-2-yl)phenyl)-4-isopropylbenzamide in step 10) of Example 1.

¹H-NMR (300 MHz, DMSO-d₆) δ 8.42 (s, 1H), 7.42˜7.30 (m, 2H), 7.27 (s, 1H), 7.22˜7.18 (m, 1H), 7.02˜6.97 (m, 1H), 6.79˜6.70 (m, 1H), 6.65˜6.62 (m, 1H), 6.10˜6.04 (m, 1H), 5.69˜5.62 (m, 1H), 4.45˜4.38 (m, 3H), 2.11˜2.02 (m, 1H), 1.80˜1.73 (m, 3H), 1.55˜1.51 (m, 3H), 1.11˜1.06 (m, 2H), 0.87˜0.86 (m, 2H).

MS (ESI⁺): m/z=566.23 [M+H]⁺.

### TEST EXAMPLES

### Test Example 1. Determination of Inhibitory Effect on Enzyme Activity Against BTK

To determine whether the compounds obtained in Examples above exhibit inhibitory activity against the BTK enzyme, a kinase activity inhibition assay was performed. To do this, the SelectScreen profiling service was requested to the ThermoFisher company to proceed the Z′-LYTE kinase assay. The simple test method can be summarized as follows.

The compounds prepared in Examples above were each dissolved in DMSO to prepare a 10 mM solution, which was then delivered to the ThermoFisher Scientific company. According to the protocol provided by the ThermoFisher Scientific company, the solution of each compound was diluted in 100% DMSO to a have a concentration of 1,000 nM to 0.32 nM (dilution factor of 1/5). Next, the BTK enzyme was diluted in a kinase buffer (50 mM HEPES (PH 7.5), 10 mM MgCl₂, 1 mM EGTA, 0.01% BRIJ-35) to a concentration of up to 10 ng/assay. The test was performed in a low volume 384-well plate (low volume NBS, black 384-well plate). First, 100 nL of the diluted compound solution was added to a sample, and then 2.4 μL of buffer, 5 μL of mixed solution of peptide substrate and kinase at twice (2×) the concentration listed in the protocol, and 2.5 μL of 140 μm ATP solution were added thereto, followed by stirring for 30 seconds. Then, it was allowed for a reaction at room temperature for 60 minutes. Afterwards, 5 μL of a color developing solution was added thereto and stirred for 30 seconds, and fluorescence of the peptide substrate was reacted for 60 minutes. After completion of the reaction, each fluorescence value (400 nm excitation filter, 445/520 nm emission filter) was measured by using a fluorescence plate reader. Here, according to the Z′-LYTE kinase assay protocol, the degree to which the compound inhibits a kinase reaction is calculated as a phosphorylation inhibition rate of 0% to 100% compared to the control group, and the concentration at which the 50% activity was inhibited was calculated to derive a 50% inhibitory concentration (IC₅₀) value. The GraphPad was used for the analysis of the results of each compound and calculation of IC₅₀ values, and the results are shown in Table 1 below.

TABLE 1

| Example | BTK enzyme activity (IC₅₀, nM) |
|---|---|
| 1 | 1.7 |
| 2 | 1.5 |
| 3 | 3.8 |
| 4 | 5.3 |
| 5 | 6 |
| 6 | 3 |
| 7 | 20 |
| 8 | 3.4 |
| 9 | 34 |
| 10 | 2.3 |
| 11 | 10 |
| 12 | 4.9 |
| 13 | 44 |
| 14 | 24 |
| 15 | 34 |
| 16 | 29 |
| 17 | 4.1 |
| 18 | 9.7 |
| 19 | 201 |
| 20 | 16 |
| 21 | 105 |
| 22 | 5.7 |
| 23 | 8.7 |
| 13 | 44 |
| 14 | 24 |
| 15 | 34 |
| 16 | 29 |
| 17 | 4.1 |
| 18 | 9.7 |
| 19 | 201 |
| 20 | 16 |
| 21 | 105 |
| 22 | 5.7 |
| 23 | 8.7 |

As shown in Table 1, the compounds of Examples 1 to 23 showed excellent inhibitory activity against the BTK enzyme.

1. A compound of Formula I, a solvate or stereoisomer thereof, or a pharmaceutically acceptable salt thereof: 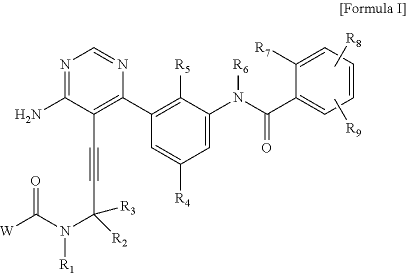
wherein, in Formula I,
W is a substituted or unsubstituted C₁-C₆ alkyl group, a substituted or unsubstituted C₂-C₆ alkenyl group, a substituted or unsubstituted C₂-C₆ alkynyl group, a substituted or unsubstituted C₁-C₆ alkoxy group, or a substituted or unsubstituted C₃-C₁₀ heterocycloalkyl group;
R₁, R₂, and R₃ are each independently hydrogen, a substituted or unsubstituted C₁-C₆ alkyl group, or a substituted or unsubstituted C₃-C₁₀ cycloalkyl group, or R₁ is linked to either R₂ or R₃ to form a 4- to 7-membered heterocyclic ring together with atoms to which they are bound;
R₆ and R₇ are each independently hydrogen, a halogen, or a substituted or unsubstituted C₁-C₆ alkyl group, or are linked to each other to form an unsaturated or partially saturated 5- to 7-membered heteroaryl ring together with atoms to which they are bound, wherein the heteroaryl ring comprises 1 to 3 hetero atoms selected from the group consisting of nitrogen (N), oxygen (O), and sulfur (S);
R₄, R₅, R₈, and R₉ are each independently hydrogen, a halogen, a cyano group, a hydroxyl group, a thiol group, a nitro group, a substituted or unsubstituted C₁-C₆ alkyl group, a substituted or unsubstituted C₂-C₆ alkenyl group, a substituted or unsubstituted C₂-C₆ alkynyl group, a substituted or unsubstituted C₁-C₆ alkoxy group, a substituted or unsubstituted C₃-C₆ cycloalkyl group, or a substituted or unsubstituted C₃-C₆ heterocycloalkyl group; and
the “substituted or unsubstituted” is being substituted with a substituent selected from a halogen, a cyano group, a hydroxyl group, a thiol group, a nitro group, a C₁-C₆ alkyl group, a C₂-C₆ alkenyl group, a C₂-C₆ alkynyl group, a C₁-C₆ alkoxy group, a C₃-C₁₀ cycloalkyl group, a C₃-C₁₀ heterocycloalkyl group, a C₃-C₁₀ aryl group, and a C₁-C₁₀ heteroaryl group or having no substituent.

2. The compound of claim 1, wherein
W is a substituted or unsubstituted C₂-C₆ alkenyl group;
R₁, R₂, and R₃ are each independently hydrogen or a substituted or unsubstituted C₁-C₆ alkyl group, or R₁ is linked to either R₂ or R₃ to form a 4- to 7-membered heterocyclic ring together with atoms to which they are bound;
R₆ and R₇ are each independently hydrogen, a halogen, or a substituted or unsubstituted C₁-C₆ alkyl group, or are linked to each other to form an unsaturated or partially saturated 6-membered heteroaryl ring together with atoms to which they are bound, wherein the heteroaryl ring comprises 1 to 3 nitrogen atoms; and
R₄, R₅, R₈, and R₉ are each independently hydrogen, a halogen, a substituted or unsubstituted C₁-C₆ alkyl group, or a substituted or unsubstituted C₃-C₆ cycloalkyl group.

3. The compound of claim 1, wherein
W is a substituted or unsubstituted C₂-C₆ alkenyl group;
R₁, R₂, and R₃ are each independently hydrogen or a substituted or unsubstituted C₁-C₃ alkyl group, or R₁ is linked to either R₂ or R₃ to form a 4- to 7-membered azacyclic ring together with atoms to which they are bound;
R₄ and R₅ are each independently hydrogen, a halogen, or a substituted or unsubstituted C₁-C₃ alkyl group;
R₆ and R₇ are each independently hydrogen, a halogen, or a substituted or unsubstituted C₁-C₃ alkyl group, or are linked to each other to form an unsaturated or partially saturated 6-membered heteroaryl ring together with atoms to which they are bound, wherein the heteroaryl ring comprises 1 to 3 nitrogen atoms;
R₈ is hydrogen, a halogen, a substituted or unsubstituted C₁-C₆ alkyl group, or a substituted or unsubstituted C₃-C₆ cycloalkyl group; and
R₉ is hydrogen, a halogen, or a substituted or unsubstituted C₁-C₃ alkyl group.

4. The compound of claim 1, wherein
W is 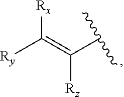
Rₓ, R_{y}, and R_{z} are each independently hydrogen, a halogen, a substituted or unsubstituted C₁-C₆ alkyl group, a cyano group, or —(CH₂)ₙ—NR₁₀R₁₁;
n is an integer from 0 to 6; and
R₁₀ and R₁₁ are each independently a substituted or unsubstituted C₁-C₆ alkyl group, or are linked to each other to form a 4- to 7-membered azacyclic ring together with nitrogen (N) atom to which they are bound.

5. The compound of claim 4, wherein
Rₓ is hydrogen, a halogen, or a substituted or unsubstituted C₁-C₃ alkyl group;
R_{y} is hydrogen, a halogen, a substituted or unsubstituted C₁-C₃ alkyl group, or —(CH₂)ₙ—NR₁₀R₁₁;
R_{z} is hydrogen, a halogen, a substituted or unsubstituted C₁-C₆ alkyl group, or a cyano group;
n is an integer of 0 or 1; and
R₁₀ and R₁₁ are each independently a substituted or unsubstituted C₁-C₃ alkyl group, or are linked to each other to form a 4- to 7-membered azacyclic ring together with nitrogen (N) atom to which they are bound.

6. The compound of claim 1, wherein the compound of Formula I is a compound of Formula Ia below: 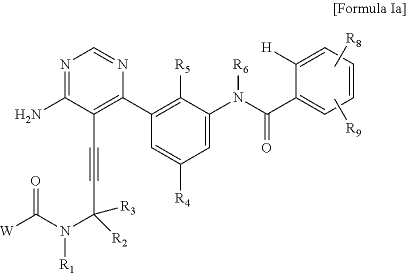
wherein, in Formula Ia, W, R₁, R₂, R₃, R₄, R₅, R₆, R₈, and R₉ are as defined in claim 1.

7. The compound of claim 1, wherein the compound of Formula I is a compound of Formula Ib below: 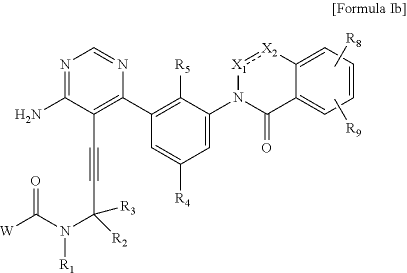
wherein, in Formula Ib,
X₁ and X₂ are each independently —C(R₁₂)₂—, —CR₁₂—, or —N—;
‘ [symbol] ’ is a single bond or a double bond;
R₁₂ is hydrogen or a substituted or unsubstituted C₁-C₆ alkyl group; and
W, R₁, R₂, R₃, R₄, R₅, R₈, and R₉ are as defined in claim 1.

8. The compound of claim 1, wherein the compound of Formula I is selected from the group consisting of compounds of 1) to 23):
1) N-(3-(6-amino-5-(3-N-methylacrylamido)prop-1-yn-1-yl)pyrimidin-4-yl)5-fluoro-2-methylphenyl)-2-fluoro-4-isopropylbenzamide;
2) N-(3-(6-amino-5-(3-N-methylacrylamido)prop-1-yn-1-yl)pyrimidin-4-yl)5-fluoro-2-methylphenyl)-4-cyclopropyl-2-fluorobenzamide;
3) N-(3-(4-amino-6-(3-(6-cyclopropyl-8-fluoro-1-oxo-3,4-dihydroisoquinolin-2(1H)-yl)-5-fluorophenyl)pyrimidin-5-yl)prop-2-yn-1-yl)-N-methylacrylamide;
4) N-(3-(4-amino-6-(3-(6-cyclopropyl-8-fluoro-1-oxo-3,4-dihydroisoquinolin-2(1H)-yl)-5-fluoro-2-methylphenyl)pyrimidin-5-yl)prop-2-yn-1-yl)-N-methylacrylamide;
5) N-(3-(4-amino-6-(3-(6-cyclo-8-fluoro-1-oxoisoquinolin-2(1H)-yl)-5-fluorophenyl)pyrimidin-5-yl)prop-2-yn-1-yl)-N-methylacrylamide;
6) N-(3-(4-amino-6-(3-(6-cyclopropyl-8-fluoro-1-oxoisoquinolin-2(1H)-yl)-5-fluoro-2-methylphenyl)pyrimidin-5-yl)prop-2-yn-1-yl)-N-methylacrylamide;
7) N-(3-(4-amino-6-(3-(6-cyclopropyl-8-fluoro-1-oxoisoquinolin-2(1H)-yl)-2,5-difluorophenyl)pyrimidin-5-yl)prop-2-yn-1-yl)-N-methylacrylamide;
8) N-(3-(4-amino-6-(3-(6-cyclopropyl-8-fluoro-1-oxoisoquinolin-2(1H)-yl)-2,5-dimethylphenyl)pyrimidin-5-yl)prop-2-yn-1-yl)-N-methylacrylamide;
9) N-(3-(4-amino-6-(3-(6-cyclopropyl-8-fluoro-1-oxoisoquinolin-2(1H)-yl)-5-fluoro-2-methylphenyl)pyrimidin-5-yl)prop-2-yn-1-yl)acrylamide;
10) N-(3-(4-amino-6-(3-(6-(tert-butyl)-8-fluoro-1-oxophthalazin-2-(1H)-yl)-5-fluorophenyl)pyrimidin-5-yl)prop-2-yn-1-yl)-N-methylacrylamide;
11) N-(3-(4-amino-6-(3-(6-cyclopropyl-1-oxophthalazin-2(1H)-yl)-5-fluoro-2-methylphenyl)pyrimidin-5-yl)prop-2-yn-1-yl)-N-methylacrylamide;
12) N-(3-(4-amino-6-(3-(6-cyclopropyl-8-fluoro-1-oxophthalazin-2(1H)-yl)-5-fluoro-2-methylphenyl)pyrimidin-5-yl)prop-2-yn-1-yl)-N-methylacrylamide;
13) N-(3-(4-amino-6-(3-(6-(tert-butyl)-8-fluoro-1-oxophthalazin-2(1H)-yl)-5-fluoro-2-methylphenyl)pyrimidin-5-yl)prop-2-yn-1-yl)-N-methylacrylamide;
14) N-(3-(4-amino-6-(3-(6-tert-butyl)-1-oxophthalazin-2-(1H)-yl)-5-fluorophenyl)pyrimidin-5-yl)prop-2-yn-1-yl)-N-methylacrylamide;
15) N-(3-(4-amino-6-(3-(7-cyclopropyl-5-fluoro-4-oxoquinazolin-3(4H)-yl)-5-fluorophenyl)pyrimidin-5-yl)prop-2-yn-1-yl)-N-methylacrylamide;
16) (R)-2-(3-(5-((1-acryloylpyrrolidin-2-yl)ethynyl)-6-aminopyrimidin-4-yl)-5-fluoro-2-methylphenyl)-6-cyclopropyl-8-fluoroisoquinolin-1(2H)-one;
17) (S)-2-(3-(5-((1-acryloylpyrrolidin-2-yl)ethynyl)-6-aminopyrimidin-4-yl)-5-fluoro-2-methylphenyl)-6-cyclopropyl-8-fluoroisoquinolin-1(2H)-one;
18) (S)-2-(3-(5-((1-acryloylpyrrolidin-2-yl)ethynyl)-6-aminopyrimidin-4-yl)-5-fluoro-2-methylphenyl)-8-fluoro-6-isoquinolin-1-(2H)-one;
19) (S)-2-(3-(6-amino-5-((1-(2-fluoroacryloyl)pyrrolidin-2-yl)ethynyl)pyrimidin-4-yl)-5-fluoro-2-methylphenyl)-6-cyclopropyl-8-fluoroisoquinolin-1(2H)-one;
20) (S)-2-(3-(6-amino-5-((1-(2-chloroacryloyl)pyrrolidin-2-yl)ethynyl)pyrimidin-4-yl)-5-fluoro-2-methylphenyl)-6-cyclopropyl-8-fluoroisoquinolin-1(2H)-one;
21) (S)—N-(3-(5-((1-acryloylpyrrolidin-2-yl)ethynyl)-6-aminopyrimidin-4-yl)-5-fluoro-2-methylphenyl)-4-cyclopropyl-2-fluorobenzamide;
22) 2-(3-(5-((1-acryloylpiperidin-2-yl)ethynyl)-6-aminopyrimidin-4-yl)-5-fluoro-2-methylphenyl)-6-cyclopropyl-8-isoquinolin-1(2H)-one; and
23) (S)-2-(3-(5-((1-acryloylpiperidin-2-yl)ethynyl)-6-aminopyrimidin-4-yl)-5-fluoro-2-methylphenyl)-8-fluoro-6-isopropylisoquinolin-1(2H)-one.

9. A method for preventing or treating Bruton's tyrosine kinase (BTK)-mediated disease in a subject, the method comprising administering to the subject a pharmaceutical composition comprising the compound of claim 1,
wherein the BTK-mediated disease is autoimmune disease or cancer.

10. The method of claim 9, wherein the pharmaceutical composition has inhibitory activity against BTK.

11. The method of claim 9, wherein the autoimmune disease is any one selected from the group consisting of rheumatic arthritis, psoriasis arthritis, osteoarthritis, Still's disease, juvenile arthritis, lupus, myasthenia gravis, Hashimoto's thyroiditis, iodide thyroiditis, Graves' disease, Sjogren's syndrome, multiple sclerosis, Guillain-Barre syndrome, acute disseminated encephalomyelitis, Addison's disease, nystagmus-myoclonus syndrome, ankylosing spondylitis, antiphospholipid antibody syndrome, aplastic anemia, autoimmune hepatitis, celiac disease, Goodpasture's syndrome, idiopathic thrombocytopenic purpura, optic neuritis, scleroderma, primary biliary cirrhosis, Reiter's syndrome, Takayasu arteritis, temporal arteritis, warm autoimmune hemolytic anemia, Wegener's granulomatosis, psoriasis, alopecia universalis, Behcet's disease, autonomic dysfunction, asthma, chronic spontaneous urticaria, pemphigus, systemic lupus erythematosus, hidradenitis, skin disease, immunoglobulin G4-related disease, endometrioma, interstitial cystitis, neuromyotonia, and vulvodynia.

12. The method of claim 9, wherein the cancer is B-cell malignancy.

13. The method of claim 9, wherein the pharmaceutical composition is formulated in the form of tablets, pills, powders, capsules, syrups, emulsions, or microemulsions.

14. (canceled)

15. (canceled)

16. A pharmaceutical composition comprising a compound of claim 1 and a pharmaceutically acceptable carrier.
